(19) United States
(12) Patent Application Publication
(10) Pub. No.: US 2026/0237534 A1
(43) Pub. Date: Aug. 13, 2026

(54) RADIOISOTOPE SOURCE

(71) Applicant: ADVANCELL PTY LIMITED, Sydney (AU)

(72) Inventors: Nicolò CANEVER, Sydney (AU); Julian KELLY, Sydney (AU); Simon PUTTICK, Sydney (AU); William TIEU, Sydney (AU); Kevin KUAN, Sydney (AU)

(21) Appl. No.: 19/157,479

(22) PCT Filed: Feb. 20, 2024
(86) PCT No.: PCT/AU2024/050123

§ 371 Date: Aug. 18, 2025

(30) Foreign Application Priority Data
Feb. 20, 2023 (AU) 2023900421

(51) Int. Cl. G21G 4/08; A61K 51/00; G21G 1/00; G21G 1/04

(52) U.S. Cl. CPC G21G 4/08; A61K 51/00; G21G 1/0005; G21G 1/001; G21G 1/04

(57) ABSTRACT

A layered radioisotope source for generating a medically useful dose of daughter radioisotope through a chain of spontaneous decay from a parent radioisotope via a gaseous intermediate radioisotope, comprising: an inert ceramic substrate layer; a metal oxide surface layer bound on the inert ceramic substrate layer; and parent radioisotope immobilised on or within the metal oxide surface layer to allow for effective emanation of the gaseous intermediate radioisotope away from the layered radioisotope source.

### FIELD

The present disclosure generally relates to a radioisotope source, and more particularly to a layered radioisotope source comprising at least an inert ceramic substrate layer and a metal oxide surface layer having radioisotope immobilised therein, processes for preparing the layered radioisotope source, and processes, generators, and systems, and the like, for the producing and capturing radioisotope using the layered radioisotope source, including the preparation of radioisotope solutions for use in radiopharmacy and/or in other clinical applications.

### BACKGROUND

Radioisotopes have a variety of uses including, for example, in medical applications as radiopharmaceuticals in which radioisotopes can effectively deliver lethal radiation directly to cancer cells with little collateral damage to surrounding healthy tissue. However, clinical uptake of this form of therapy is restricted by the availability of suitable radioisotopes and global supply is limited due to various manufacturing constraints, with the current supply of alpha-emitting radioisotope worldwide being only enough to support a small number of early stage trials. Moreover, radioisotopes generated by current methods are often contaminated with impurities, including radiochemical impurities, which are difficult to filter or remove from the desired radioisotope, which can hinder clinical application.

A small number of alpha-emitting radioisotopes are produced for therapeutic/clinical trial use, for example actinium-225 (²²⁵Ac) which can be labelled to cancer targeting molecules. However, generating these isotopes in clinically useful amounts requires complex operations, including for example large particle accelerators or nuclear reactors. The supply chains for these radioisotopes are therefore unwieldy, expensive and consequently are limited to just a few manufacturing facilities worldwide.

Lead-212 (²¹²Pb) is an excellent alpha-emitting radioisotope for radioligand therapy. Current generators for producing ²¹²Pb are column based generators that employ parent radioisotope having relatively short half-life e.g. radium-224 (²²⁴Ra), which elute ²¹²Pb from parent radioisotope bound on resin-based ion exchange materials. Such resin-based ion exchange based generators are subject to significant radiolytic damage which limits their overall durability and limits clinical application. Furthermore, the extraction of the isolated ²¹²Pb radioisotope from such resin-based ion exchange based generators often requires significant amount of wash fluid thus complicating and lengthening subsequent radiolabelling chemistry processes. Additionally, most ²¹²Pb generators experience significant yield decrease over time due to radiolytic breakdown of the organic material, such as barium stearate, used to immobilise and house the parent radioisotope and/or require overly complex loading procedures for immobilising the parent radioisotope which can expose users to significant radiation doses.

Accordingly, there is a need for improved radioisotope sources and processes for generating therapeutic radioisotopes, which can allow for the production of clinically useful doses of therapeutic isotope.

### SUMMARY

The present inventors have undertaken research and development into identifying new and improved sources for medical radioisotope which address one or more of the above problems, or at least provide the public with a useful alternative. In particular, the present inventors have developed a layered radioisotope source that can be configured to immobilise parent radioisotope which can be used as a source for generating daughter radioisotope.

According to some embodiments or examples described herein, the present inventors have developed a “sol-gel” process capable of providing a substantially homogenous dispersion of radioisotope immobilised within the surface layer of an ceramic substrate. Importantly and unlike other deposition and precipitation methods which can form large discrete particles/deposits of radioisotope loosely adhered to the substrate surface (e.g. wool or paper substrates comprising loose deposits of radioisotope which are difficult to handle due to potential contamination), not only is the immobilised radioisotope strongly anchored/embedded within the surface layer, but in some embodiments the surface layer itself is tightly bound to the underlying ceramic substrate. Owing to these properties, the layered radioisotope source is expected to have one or more further advantages over existing sources, including high resistance to radiolytic damage, high radionuclidic product purity due to lower parent radioisotope breakthrough into the collected daughter radioisotope, high production efficiency due to the effective separation of gaseous intermediate radioisotope as it decays and emanates away from the immobilised parent radioisotope and/or longer lifespan of the source. These advantageous properties enabled the development of a radioisotope generator that allowed for “line-of-sight” gravity assisted collection of daughter radioisotope with minimal contamination.

In one aspect, there is provided a layered radioisotope source for generating a medically useful dose of daughter radioisotope through a chain of spontaneous decay from a parent radioisotope via a gaseous intermediate radioisotope, comprising:
an inert ceramic substrate layer;
a metal oxide surface layer bound on the inert ceramic substrate layer; and
parent radioisotope immobilised on or within the metal oxide surface layer to allow for effective emanation of the gaseous intermediate radioisotope away from the layered radioisotope source.

In another aspect, there is provided a sol-gel process for preparing a layered radioisotope source for generating a medically useful dose of daughter radioisotope through a chain of spontaneous decay from a parent radioisotope via a gaseous intermediate radioisotope, comprising:
a) providing on the surface of an inert ceramic substrate layer a gel formed from a solution (e.g. sol) comprising a metal alkoxide and a parent radioisotope species:
b) heating the gel under conditions effective to form a metal oxide surface layer bound on the inert ceramic substrate layer,
wherein the parent radioisotope is immobilised on or within the metal oxide surface layer to allow for effective emanation of the gaseous intermediate radioisotope away from the layered radioisotope source.

In another aspect, there is provided a radioisotope generator defining a chamber for producing and capturing a population of daughter radioisotope, wherein the chamber is configured to house a layered radioisotope source as described herein in the chamber. In another aspect, there is provided a radioisotope generator defining a chamber for producing and capturing a population of daughter radioisotope, wherein the chamber houses a layered radioisotope source as described herein in the chamber.

In another aspect, there is provided a system for producing and capturing a population of daughter radioisotope, comprising:
a) a radioisotope generator defining a chamber for producing and capturing a population of daughter radioisotope described herein; and
b) a layered radioisotope source described herein housed in the chamber.

In another aspect, there is provided process for producing and capturing a population of daughter radioisotope comprising:
a) allowing for the emanation of a gaseous intermediate radioisotope generated through a chain of spontaneous decay from a parent radioisotope immobilised on or within a layered radioisotope source described herein; and
b) collecting at least some of the gaseous intermediate radioisotope for a period of time effective for it to decay into a daughter radioisotope.

Other aspects and embodiments relating to the present disclosure are described herein. It will be appreciated that each example, aspect and embodiment of the present disclosure described herein is to be applied mutatis mutandis to each and every other example, aspect or embodiment unless specifically stated otherwise. For example, each example, aspect and embodiment of the layered radioisotope source described herein may apply equally to one or more of the generator, system or processes described herein, and vice versa. The present disclosure is not to be limited in scope by the specific examples described herein, which are intended for the purpose of exemplification only. Functionally-equivalent products, compositions and processes are clearly within the scope of the disclosure as described herein.

### BRIEF DESCRIPTION OF FIGURES

Embodiments of the present disclosure are further described and illustrated as follows, by way of example only, with reference to the accompanying drawings in which:

FIG. 1: Schematic of one embodiment of an inert ceramic substrate comprising immobilised radioisotope.

FIGS. 2 to 17: SEM micrographs and related EDX region scan spectra of layered radioisotope sources prepared according to Table 1.

FIG. 18: A radioactive decay series for thorium-228 (²²⁸Th) which in some embodiments comprise the radioisotope of the present disclosure.

### DETAILED DESCRIPTION

The present disclosure describes the following various non-limiting embodiments, which relate to investigations undertaken to develop radioisotope sources, processes, generators, and/or systems, for generating radioisotope.

### Terms

In the following description, reference may made to the accompanying figures and/or drawings which form a part hereof, and which is shown, by way of illustration, several embodiments. It is understood that other embodiments may be utilized and structural changes may be made without departing from the scope of the present disclosure.

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, processes, substrates, etc. referred to or indicated in this specification, individually or collectively, and any and all combinations or any two or more of said steps or features.

In describing examples and embodiments, herein, specific terminology is used for the sake of clarity. For the purpose of description, specific terms are intended to at least include technical and functional equivalents that operate in a similar manner to accomplish a similar result. Additionally, in some instances where a particular embodiment or example includes a plurality of system elements or method steps, those elements or steps may be replaced with a single element or step. Likewise, a single element or step may be replaced with a plurality of elements or steps that serve the same purpose.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

Where methods/processes are recited and where steps/stages are recited in a particular order—with or without sequenced prefacing characters added for ease of reference—the steps/stages are not to be interpreted as being temporally limited to the order in which they are recited unless otherwise specified or implied by the terms and phrasing.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “about”, unless stated to the contrary, typically refers to +/−10%, for example +/−5%, of the designated value.

Where parameters for various properties or other values are specified herein for examples or embodiments, those parameters or values can be adjusted up or down by 1/100th, 1/50th, 1/20th, 1/10th, ⅕th, ⅓rd, ½, ⅔rd, ¾th, ⅘th, 9/10th, 19/20th, 49/50th, 99/100th, etc. (or up by a factor of 1, 2, 3, 4, 5, 6, 8, 10, 20, 50, 100, etc.), or by rounded-off approximations thereof or within a range of the specified parameter up to or down to any of the variations specified above (e.g., for a specified parameter of 100 and a variation of 1/100th, the value of the parameter may be in a range from 0.99 to 1.01), unless otherwise specified.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various components/features of the disclosure can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 4.5 and 5, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The phrase “consisting of” means the enumerated elements and no others.

As used herein, the term “decay” refers to the spontaneous transformation of a radioactive nuclide into a different nuclide referred to as its daughter, or its “decay product”. The daughter nuclide may be stable or it may itself be radioactive and will thus undergo further spontaneous decay into a different daughter nuclide. It is understood that these radioactive decay processes occur spontaneously without the need for human intervention.

As used herein, the term “isotope” and “nuclide”, including for example when used in compound words such as radioisotope and radionuclide, are synonymous and can be used interchangeably.

### Layered Radioisotope Sources

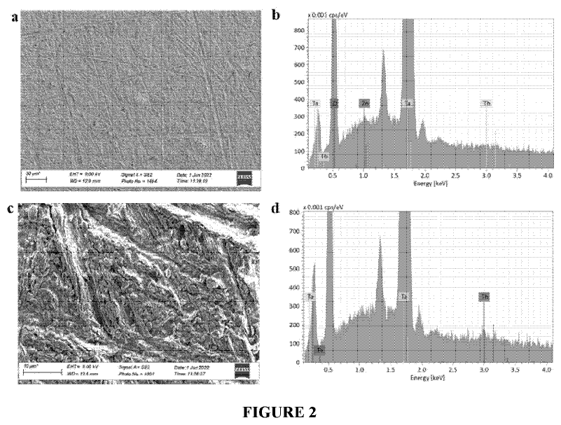

The present inventors have developed a layered radioisotope source that can be configured to immobilise parent radioisotope which can be used as a source for generating daughter radioisotope, including those provided in the decay series in FIG. 2, such as ²¹²Pb. The parent radioisotope can be used as a source for a gaseous intermediate radioisotope which can be captured and used in turn as a source for generating a daughter radioisotope using the processes and generators described herein.

According to some embodiments or examples described herein, it was found that by strongly anchoring/embedding the parent radioisotope within a metal oxide surface layer tightly adhered to an underlying inert ceramic substrate, the resulting layered radioisotope source is expected to have one or more further advantages including high resistance to radiolytic damage, increased daughter radioisotope yield/purity, and/or a longer lifespan.

In one embodiment, there is provided a layered radioisotope source, comprising: an inert ceramic substrate layer; a metal oxide surface layer bound on the inert ceramic substrate layer; and parent radioisotope immobilised on or within the metal oxide surface layer.

The layered radioisotope source may have a morphology suitable for inserting into a radioisotope generator, for example into the source chamber described herein. In one embodiment, the layered radioisotope source may be provided as a discrete unit, for example a disk (e.g. cylindrical), plate, film, platen, slab, tube, tube-section or monolith. The unit may have any desired shape including, but not limited to spherical or semi-spherical. In one embodiment, the inert ceramic substrate is a disk or slab. The disk or slab may be configured to be inserted into a radioisotope generator, including for example in an “upside down” configuration relative to an external viewer. Provided the slab or disk is substantially planar in geometry, the disk is not limited to being any particular cross-sectional shape (i.e. spherical, rectangular etc.).

In one embodiment, the layered radioisotope source has a cross-sectional aspect ratio (i.e. the ratio of a length to a width, where the length and width are measured perpendicular to one another, and the length refers to the longest linear dimension) of greater than 1.0 to about 10.0, for example at least about 2.0, 2.5 3.0. 3.5. 4.0. 5.0. 6.0. 7.0. 8.0. 9.0 or 10.0. According to some embodiments or examples described herein, a layered radioisotope source having a higher cross-sectional aspect ratio (e.g. greater than 2.0) provides a planar geometry, such as a disk or slab, that provides a larger radioisotope surface layer area for effective emanation of gaseous intermediate radioisotope.

In one embodiment, the layered radioisotope source has an overall thickness of between about 1 mm to about 100 mm. The layered radioisotope source may have a thickness (in mm) of at least about 0.5, 1, 2, 3, 4, 5, 6, 7, 8, 9, 10, 12, 15, 20, 25, 30, 40, 50, or 100. The layered radioisotope source may have a thickness (in mm) of less than about 100, 50, 40, 30, 25, 20, 15, 12, 10, 9, 8, 7, 6, 5, 4, 3, 2, 1 or 0.5. The thickness may be a range provided by any two of these upper or lower values, for example between about 1 mm to about 100 mm, between about 1 mm to about 20 mm, or between about 1 mm to about 15 mm, e.g. between about 1 mm to about 10 mm.

### Types of Radioisotope

Any suitable parent radioisotope may be used. In one embodiment, the parent radioisotope is an alpha-emitting radioisotope i.e. capable of emitting an alpha particle (i.e. helium nucleus) and thereby transforms into a different atomic nucleus with a mass number that is reduced by four and an atomic number that is reduced by two.

In one embodiment, the parent radioisotope is an isotope of thorium or radium or a combination thereof. In one embodiment, the parent radioisotope is a thorium radioisotope. The thorium radioisotope may be selected from at least one of thorium-227 (²²⁷Th), thorium-228 (²²⁸Th), and thorium-232 (²³²Th), or combination thereof. In one embodiment, the parent radioisotope is radium. The radium radioisotope may be selected from at least one of ²²⁴Ra and ²²⁸Ra, or a combination thereof.

In one embodiment, the parent radioisotope is ²²⁸Th. ²²⁸Th has a half-life of almost two years and is available commercially. These and other properties of ²²⁸Th make it desirable as a parent radioisotope for producing ²¹²Pb. ²¹²Pb is a promising medical isotope for targeted therapy including for cancer treatment, as it decays to α-particle emitter ²¹²Bi via β-particle emission, thus extending the problematic short half-life of ²¹²Bi. When ²¹²Pb is coupled to targeting molecules such as those for cancer cells, it can be used in Targeted Alpha Therapy.

Once immobilised within the layered radioisotope source described herein, according to some embodiments or examples, ²²⁸Th can be used as a parent radioisotope in a ²¹²Pb generator for a year or more with only gradual loss of productivity.

In one embodiment, the gaseous intermediate radioisotope is a radon radioisotope. The radon radioisotope may be selected from at least one of radon-219 (²¹⁹Rn) or radon-220 (²²⁰Rn). In one embodiment, the daughter radioisotope is a lead radioisotope. The lead radioisotope may be selected from at least one of lead-211 (²¹¹Pb) or lead-212 (²¹²Pb).

In one embodiment, the parent radioisotope are immobilised on or within the metal oxide surface layer in a manner that allows for effective emanation of gaseous intermediate radioisotope away from the layered radioisotope source.

In one embodiment, the parent radioisotope are immobilised on or within the metal oxide surface layer in an amount effective to generate a medically useful dose of daughter radioisotope through a chain of spontaneous decay of a gaseous intermediate radioisotope. Similarly, in one embodiment, the layered radioisotope source is for generating a medically useful dose of daughter radioisotope through a chain of spontaneous decay from a parent radioisotope via a gaseous intermediate radioisotope.

As used herein, the term “medically useful” in relation to the dose of daughter radioisotope refers to an amount of daughter radioisotope (e.g. ²¹²Pb) that can be used as a product for radiopharmacy applications such as radioligand therapy, e.g. Targeted Alpha Therapy. The parent radioisotope may be provided in an amount effective to generate a medically useful amount (e.g. pre-clinically and/or clinically useful amount) of daughter radioisotope.

In one embodiment, the parent radioisotope may be immobilised on or within the metal oxide surface layer bound on the inert ceramic substrate layer in an amount effective to generate a medical dose or a preclinical study dose of daughter radioisotope (e.g. ²¹²Pb) of between about 1 to about 1,000 MBq. In one embodiment, the parent radioisotope may be immobilised on or within the metal oxide surface layer bound on the inert ceramic substrate layer in an amount effective to generate a medical dose or a preclinical study dose of daughter radioisotope (e.g. ²¹²Pb) of at least about 1, 2, 5, 10, 50, 60, 90, 120, 150, 200, 250, 300, 400, 500, 600, 700, 800, 900 or 1,000 MBq. In another embodiment, the parent radioisotope may be immobilised on or within the metal oxide surface layer bound on the inert ceramic substrate layer in an amount effective to generate a medical dose or a preclinical study dose of daughter radioisotope (e.g. ²¹²Pb) of less than about 1,000, 900, 800, 700, 600, 500, 400, 300, 250, 200, 150, 120, 90, 60, 50, 10, 5, 2 or 1. In one embodiment, the parent radioisotope may be immobilised on or within the metal oxide surface layer bound on the inert ceramic substrate layer in an amount effective to generate a medical dose of daughter radioisotope (e.g. ²¹²Pb) of at least about 50, 70, 100, 120, 140, 160, 180 or 200 MBq. The parent radioisotope may be immobilised on or within the metal oxide surface layer bound on the inert ceramic substrate layer in an amount effective to generate a medical dose of daughter radioisotope (e.g. ²¹²Pb) in a range provided by any two of these upper and/or lower values, for example between about 50 MBq to about 200 MBq.

In one embodiment, the parent radioisotope immobilised on or within the metal oxide surface layer bound on the inert ceramic substrate layer is present in an amount effective to provide an activity (in MBq per cm² of metal oxide surface) of between about 1 to about 1500. The parent radioisotope may be immobilised on or within the metal oxide surface layer bound on the inert ceramic substrate layer in an amount effective to provide an activity (in MBq per cm² of metal oxide surface) of at least about 0.01, 0.05, 1, 2, 5, 10, 20, 50, 70, 100, 200, 300, 400, 500, 600, 700, 800, 900, 1000, 1200, or 1500. The parent radioisotope may be immobilised on or within the metal oxide surface layer bound on the inert ceramic substrate layer in an amount effective to provide an activity (in MBq per cm² of metal oxide surface) of less than about 1500, 1200, 1000, 900, 800, 700, 600, 500, 400, 300, 200, 100, 70, 50, 20, 10, 5, 2, 1, 0.05 or 0.01. The parent radioisotope may be immobilised on or within the metal oxide surface layer bound on the inert ceramic substrate layer in an amount effective to provide an activity in a range provided by any two of these upper and/or lower values, for example between about 100 to about 1500, between about 10 to about 1000, or between about 50 to about 500, for example about 100. The activity of the parent radioisotope immobilised on or within the metal oxide surface layer may be measured using a suitable radioactivity measurement apparatus, or by inference from the amount of daughter radioisotope/s collected at a distance from the substrate. Activity can also be obtained via suitable simulation and modelling.

Immobilisation of Parent Radioisotope within the Metal Oxide Surface Layer

The parent radioisotope is immobilised on or within the metal oxide surface layer of the layered radioisotope source. In one embodiment, the layered radioisotope source comprises parent radioisotope immobilised on or within the metal oxide surface layer. For example, the parent radioisotope may be fixed/bound within the metal oxide surface layer.

The immobilised parent radioisotope may be interspersed within the metal oxide surface layer. The immobilised parent radioisotope may be interspersed within the lattice of the metal oxide surface layer. The parent radioisotope may be incorporated or embedded within the metal oxide surface layer. By immobilising the parent radioisotope within the metal oxide surface layer, parent radioisotope atoms are not able to migrate from the surface, yet intermediate daughter radioisotope that is gaseous can emanate and diffuse away from the layered radioisotope source to provide effective separation of the emanated daughter radioisotope from the immobilised parent radioisotope.

In one embodiment, the parent radioisotope is immobilised on or within the metal oxide surface layer in a manner that allows for effective emanation of the gaseous intermediate radioisotope away from the layered radioisotope source.

In one embodiment, the metal oxide surface layer is a heat-treated metal oxide surface layer. As used herein, the term “heat-treated” metal oxide surface layer refers to a surface layer which has been formed via thermal treatment (i.e. heated) to both chemically and/or physically bind parent radioisotope therein but also strongly adhere the metal oxide surface layer to the underlying inert ceramic substrate layer.

In one embodiment, at least some of the metal oxide surface layer is a calcined metal oxide surface layer. As used herein, the term “calcined” metal oxide surface layer refers to at least a portion of the surface of metal oxide surface layer which has been formed via the thermal treatment (i.e. heating) of a metal oxide precursor, for example a gel e.g. a xerogel or aerogel formed via a sol-gel process, so that it is oxidized, reduced or loses one or more volatile substances, forming the metal oxide surface layer.

The calcined metal oxide surface layer may be obtainable by a process comprising: a) providing on the surface of an inert ceramic substrate layer a gel formed from a solution (e.g. sol) comprising a metal alkoxide and a parent radioisotope species; b) heating (e.g. calcining) the gel under conditions effective to form a metal oxide surface layer bound on the inert ceramic substrate layer, thereby forming a calcined metal oxide surface layer comprising immobilised parent radioisotope to allow for effective emanation of the gaseous daughter radioisotope away from the inert ceramic substrate.

In a related embodiment, the metal oxide surface layer may be a sol-gel reaction product. For example, the metal oxide surface layer may be a reaction product of a sol-gel process described herein. In another related embodiment, the metal oxide surface layer may be a sol-gel derived metal oxide surface layer. That is, the metal oxide surface layer is derived from a sol-gel process described herein.

The calcined metal oxide surface layer and/or sol-gel reaction product may be obtainable by a process described herein under the section heading “Process for preparing layered radioisotope source.”

According to some embodiments or examples described herein, the present inventors have identified that the immobilised parent radioisotope within the calcined metal oxide surface layer is tightly bound/embedded therein and uniformly distributed within the calcined metal oxide surface layer. Additionally, in some embodiments, the calcined metal oxide surface layer can effectively “fuse” with the underlying inert ceramic substrate layer, and in most cases be provided as a thin and tightly bound surface layer comprising the uniformly distributed immobilised parent radioisotope. Such calcination allows for little to no parent radioisotope co-emanating with the gaseous intermediate radioisotope as the latter emanates away from the calcined metal oxide surface layer. In other words, the parent radioisotope immobilised within the calcined metal oxide surface layer spontaneously decays into gaseous intermediate radioisotope which emanates away from the tightly bound calcined metal oxide surface layer.

In one embodiment, the immobilisation of parent radioisotope within the metal oxide surface layer bound on the inert ceramic substrate layer is such that, in use, it enables the capture of a population of daughter radioisotope having a contamination level of parent radioisotope of less than about 5, 2, 1, 0.1, 0.01 or 0.001% expressed in activity terms relative to the activity of the daughter radioisotope. An example of a contaminant is ²²⁸Th in the ²¹²Pb produced using the layered radioisotope source. For example, even if the metal oxide surface layer comprising immobilised parent radioisotope comes into contact with a collection surface (e.g. the inner walls of a collection chamber) within a radioisotope generator, little to no cross-contamination of the parent radioisotope occurs owing to the metal oxide surface layer tightly binding the parent radioisotope immobilised therein, and also fusing/adhering with the underlying inert ceramic substrate layer.

The tight binding of the parent radioisotope immobilised within the metal oxide surface layer can be quantified. In one embodiment, the metal oxide surface layer comprising immobilised parent radioisotope retains at least 20%, 25%, 30%, 35%, 40%, 45%, 50%, 55%, 60%, 65%, 70%, 75%, 80%, 85%, 90%, 95% or more of the parent radioisotope after submersion in 0.1 M HNO₃. The % retention of parent radioisotope may also be in a range provided by any two of these lower values, for example the metal oxide surface layer comprising immobilised parent radioisotope retains between about 30% to about 90% of the parent radioisotope after submersion in 0.1 M HNO₃. The % retention of parent radioisotope can be measured using alpha spectroscopy.

According to some embodiments or examples described herein, it has been identified that a metal oxide surface layer prepared using the sol-gel process described herein is substantially free of detectable discrete deposits of the radioisotope. The term “discrete deposits” in relation to the radioisotope refers to the fusing/agglomeration of radioisotope into discrete particles, islands or phases of the radioisotope, for example crystalline or amorphous particles of radioisotope (including oxide particles) on or within the surface of the metal oxide surface layer. The presence of discrete deposits of radioisotope may impede to some degree the emanation of gaseous intermediate radioisotope and/or limit the sources shelf life, as such discrete deposits (e.g. in the form of large oxide particles) are more likely to hinder the release of decay products, and the poor adhesion of the deposits to the underlying substrate may lead to increased parent radioisotope breakthrough and lowered daughter radioisotope yield over the lifetime of the source.

In some embodiments, the metal oxide surface layer comprises less than about 1000, 5000, 1000, 500, 100, 80, 60, 40, 20, 10, 5 or 1 discrete deposits of the radioisotope having a particle size of about 20 nm or more bound on or within the surface of the metal oxide surface layer per cm² of metal oxide surface layer. The number of discrete deposits of the radioisotope may be in a range provided by any two of these upper values, for example, the metal oxide surface layer comprises between about 10 to about 10000, between about 100 to about 1000, or between about 1 to about 10 discrete deposits of the radioisotope having a particle size of about 20 nm or more bound on or within the surface of the metal oxide surface layer per cm³ of metal oxide surface layer. In some embodiments, the metal oxide surface layer is substantially free of discrete deposits of the radioisotope having a particle size of about 20 nm or more bound on or within the surface of the metal oxide surface layer.

The presence or absence of detectable deposits of radioisotope may be determined by scanning electron microscopy, including for example using a Zeiss Sigma VP SEM with accelerating voltage of 15 kV, 30 μm aperture size, acquired using VP mode set to 21 Pa, and backscattered electron detector-providing for magnifications of up to 50000× allowing for qualitatively determining the smallest detectable features on or within the metal oxide surface layer to about 20 nm.

Related to the substantial absence of detectable deposits of the radioisotope on or within the metal oxide surface layer, the immobilised parent radioisotope may be uniformly distributed within the metal oxide surface layer.

In one embodiment, the metal oxide surface layer has molar ratio of metal forming the oxide of the surface layer to immobilised parent radioisotope (M:R) of between greater than 1 to about 10. In one embodiment, the metal oxide surface layer may have a M:R molar ratio of at least about 1, 1.2, 1.5, 1.8, 2, 2.5, 3, 3.5, 4, 5, 6, 8 or 10. In one embodiment, the metal oxide surface layer may have a M:R molar ratio of less than about 10, 8, 6, 5, 4, 3.5, 2, 1, 1.8, 1.5, 1.2, or 1. The M:R ratio may be in a range provided by any two of these upper and/or lower values, for example between greater than 1 to about 3. According to some embodiments or examples described herein, a metal oxide surface layer having a M:R molar ratio of greater than 1, for example between greater than 1 to about 3, achieved a uniform dispersion of parent radioisotope within the surface layer as wells as good adhesion to the underlying inert ceramic substrate layer.

One advantage of the sol-gel process developed by the present inventors is that the immobilised parent radioisotope (e.g. as atoms or small oxide particles thereof) can be embedded into the metal oxide surface layer matrix and are uniformly dispersed within one or more sections of the surface layer, and in most cases can be uniformly dispersed throughout the surface layer.

In one embodiment, the parent radioisotope immobilised on or within the metal oxide surface layer is an oxide of the radioisotope (e.g. ThO₂). In one embodiment, the metal oxide surface layer and at least some of the immobilised parent radioisotope together form one or more mixed oxide phases within the metal oxide surface layer. In one embodiment, the metal oxide surface layer and immobilised parent radioisotope form a mixed oxide surface layer on the inert ceramic substrate.

In one embodiment, the mixed oxide phase or mixed oxide surface layer has the formula RₓM_{y}O_{z}, wherein R is a parent radioisotope described herein in cationic form, M is one or more metals described herein in cationic form, 0.1≤x≤5, 1.0≤y≤20, 1.0≤z≤50, preferably wherein x<y. An example of the mixed oxide phase/layer includes a TaₓTh_{y}O_{z} phase/surface layer. In one embodiment, the mixed oxide phase or mixed oxide surface layer has the formula ThₓTa_{y}O_{(2x+2.5y)}, wherein 0.1≤x≤5, and 1.0≤y≤20, preferably wherein x<y.

### Properties of the Metal Oxide Surface Layer

The parent radioisotope described herein is immobilised within a metal oxide surface layer. As understood in the art, the term “metal oxide” refers to a solid that contains one or more metal cations in a lattice of oxide anions.

The metal oxide surface layer may be located anywhere on the inert ceramic substrate. For example, the metal oxide surface layer may be a continuous layer on the inert ceramic substrate layer. Alternatively, the metal oxide surface layer may be non-continuous with respect to the entire surface of the inert ceramic substrate layer, and may comprise two or more sections, for example where the metal oxide surface layer does not uniformly and fully cover the surface of the inert ceramic substrate layer. It will be appreciated that such non-continuous layer morphology is still considered a “surface layer” for the purposes of the present disclosure.

In some embodiments, the metal oxide surface layer (including any calcined/sol-gel derived metal oxide surface layer) may have a thickness of between about 0.1 nm to about 1,000,000 nm (i.e. 1 mm), between about 0.1 nm to about 100,000 nm (i.e. 100 μm), or between about 0.1 nm to about 1,000 nm (i.e. 1 μm). The metal oxide surface layer may have a thickness (in nm) of at least about 0.1, 0.2, 0.4, 0.6, 0.8, 1, 1.2, 1.4, 1.6, 1.8, 2, 2.5, 3, 3.5, 4, 5, 6, 7, 8, 9, 10, 12, 14, 16, 18, 20, 30, 50, 100, 150, 200, 300, 400, 500, 700, 900, 1,000, 10,000, 100,000, or 1,000,000. The metal oxide surface layer may have a thickness (in nm) of less than about 1,000,000, 100,000, 10,000, 1,000, 900, 700, 500, 400, 300, 200, 150, 100, 50, 30, 20, 18, 16, 14, 12, 10, 9, 8, 7, 6, 5, 4, 3.5, 3, 2.5, 2, 1.8, 1.6, 1.4, 1.2, 1, 0.8, 0.6, 0.4, 0.2 or 0.1. The thickness may be in a range provided by any two of these upper and/or lower values, for example between about 0.1 nm to about 1,000,000 nm, between about 0.1 nm to about 100,000 nm, between about 0.1 nm to about 1,000 nm, between about 0.1 nm to about 500 nm, between about 1 nm to about 100 nm, or between about 1 nm to about 20 nm.

According to some embodiments or examples described herein, a metal oxide surface layer having a thickness of between about 0.1 nm to about 500 nm can provide further advantages, such as improved adhesion to the underlying inert ceramic substrate layer, as thicker metal oxide surface layers may have a tendency to flake and pull away from the underlying substrate. Alternatively or additionally, such sufficiently shallow thicknesses can allow for enhanced emanation of the gaseous intermediate radioisotope away from the layered radioisotope source.

The thickness of the metal oxide surface layer may vary, for example depending on the parameters of the sol-gel process described herein. For example, the metal oxide surface layer may have a thickness effective to provide a concentration of parent radioisotope in an amount effective to provide an activity (in MBq per cm² of inert ceramic substrate) as described above, for example between about 1 to about 1500.

In one embodiment, the metal oxide surface layer is an oxide of a valve metal, a refractory metal or another transition or main-block metal. The metal oxide surface layer may be an oxide on metal on which an oxide can form a continuous impervious film. The metal oxide surface layer may be an oxide of tantalum, niobium, tungsten, molybdenum, vanadium, zirconium, or titanium, or a mixed oxide thereof. In one embodiment, the metal oxide surface layer is tantalum pentoxide (Ta₂O₅). Other metals are also envisaged.

The metal oxide surface layer may have a degree of porosity effective to allow for the immobilisation of an amount of parent radioisotope yet still allow for effective emanation of gaseous daughter radioisotope away from the metal oxide surface layer, thereby providing effective separation of the emanated daughter radioisotope from the immobilised parent radioisotope. As used herein, the term “porosity” is a measure of the void spaces in a material and is a fraction of the volume of voids over the total volume as a percentage between 0 vol. % and 100 vol. %.

In some embodiments, the metal oxide surface layer has a porosity of between about 0.01 vol. % to about 30 vol. % based on the total volume of the metal oxide surface layer. The metal oxide surface layer may have a porosity (in vol. % based on the total volume of the metal oxide surface layer) of at least about 0.01, 0.1, 1, 2, 5, 10, 20, or 30. The metal oxide surface layer may have a porosity (in vol. % based on the total volume of the metal oxide surface layer) of less than about 30, 20, 10, 5, 2, 1, 0.1, or 0.01. In one embodiment, the metal oxide surface layer has a porosity (in vol. % based on the total volume of the metal oxide surface layer) of less than about 10, 5, 2, 1, 0.1, or 0.01. The porosity may be a range provided by any two of these upper and/or lower values, for example between about 10 vol. % to about 30 vol. %, or between about 0.01 vol. % to about 5 vol. %. The vol. % porosity can be measured by any suitable technique known to the person skilled in the art, including for example using standard mercury porosimetry methods and/or optical or electron microscopy analysis of a cross-section of the metal oxide surface layer. According to some embodiments or examples described herein, a metal oxide surface layer having low porosity may provide one or more advantages, including high yields of daughter isotope. For example, metal oxide surface layers having a morphology that is of low porosity, and in some cases being substantially non-porous (e.g. less than about 1, 0.1 or 0.01 vol. %) allows for more effective and substantially unimpeded emanation of gaseous intermediate isotope away from the metal oxide surface layer.

Related to porosity, the metal oxide surface layer may have low surface area, for example, a surface area (in m²/g) of less than about 200, 100, 50, 20, 10, 5, 4, 3, 2, 1 or 0.5. The surface area can be measured using standard ASTM C1274 or using N₂ adsorption with Brunauer-Emmett-Teller (BET) theory applied over the relative pressure range of 0.05 to 0.20 P/P₀ at 77 K.

The metal oxide surface layer has a suitable density, for example to provide a degree of robustness. In some embodiments, the metal oxide surface layer has a density of between about 2.0 g/cm³ to about 15 g/cm³. The metal oxide surface layer may have a density (g/cm³) of at least about 2, 3, 4, 5, 6, 7, 8, 9, 10, 11, 12, 13, 14 or 15. The metal oxide surface layer may have a density (g/cm³) of less than about 15, 14, 13, 12, 11, 10, 9, 8, 7, 6, 5, 4, 3 or 2. The density may be a range provided by any two of these upper and/or lower values, for example between about 4 to 13 g/cm³.

### Properties of the Inert Ceramic Substrate Layer

In one embodiment, there is provided a layered radioisotope source for generating a medically useful dose of daughter radioisotope through a chain of spontaneous decay from a parent radioisotope via a gaseous intermediate radioisotope, comprising:
an inert ceramic substrate layer (e.g. quartz, tantalum oxide, zirconia etc.);
a metal oxide surface layer bound on the inert ceramic substrate layer (e.g. tantalum oxide); and
parent radioisotope immobilised on or within the metal oxide surface layer to allow for effective emanation of the gaseous intermediate radioisotope away from the layered radioisotope source.

The metal oxide surface layer is bound on (e.g. bound to the surface of) an inert ceramic substrate layer. The term “inert” will be understood to mean that the ceramic substrate is substantially chemically inert, and for example does not chemically react to a significant degree with the immobilised radioisotope interspersed on or within the substrate. The inert ceramic substrate layer may also not substantially react with atmospheric oxygen and water. Additionally, the inert ceramic substrate layer may comprise ceramic material of a certain robustness making it less susceptible to radiation damage (i.e. structural damage) inflicted as the parent radioisotope immobilised therein decays into daughter radioisotope. For example, owing to the crystallographic properties of the inert ceramic substrate layer, a degree of robustness toward radiation damage is provided.

The inert ceramic substrate has a degree of porosity effective to allow for the immobilisation of an amount of parent radioisotope yet still allow for effective emanation of gaseous daughter radioisotope away from the inert ceramic substrate surface, thereby providing effective separation of the emanated daughter radioisotope from the immobilised parent radioisotope. The degree of porosity may be greater at the surface of the inert ceramic substrate. The degree of porosity may facilitate the immobilisation of a substantial amount of parent radioisotope. As used herein, the term “porosity” is a measure of the void spaces in a material and is a fraction of the volume of voids over the total volume as a percentage between 0 vol. % and 100 vol. %.

In some embodiments, the inert ceramic substrate layer has a porosity of between about 0.01 vol. % to about 30 vol. % based on the total volume of the inert ceramic substrate layer. The inert ceramic substrate layer may have a porosity (in vol. % based on the total volume of the inert ceramic substrate layer) of at least about 0.01, 0.1, 1, 2, 5, 10, 20, or 30. The inert ceramic substrate layer may have a porosity (in vol. % based on the total volume of the inert ceramic substrate layer) of less than about 30, 20, 10, 5, 2, 1, 0.1, or 0.01. In one embodiment, the inert ceramic substrate layer has a porosity (in vol. % based on the total volume of the inert ceramic substrate layer) of less than about 10, 5, 2, 1, 0.1, or 0.01. The porosity may be a range provided by any two of these upper and/or lower values, for example between about 10 vol. % to about 30 vol. %, or between about 0.01 vol. % to about 5 vol. %. The vol. % porosity can be measured by any suitable technique known to the person skilled in the art, including for example using standard mercury porosimetry methods and/or optical or electron microscopy analysis of a cross-section of the inert ceramic substrate layer.

Related to porosity, the inert ceramic substrate layer may have low surface area, for example may have a surface area (in m²/g) of less than about 200, 100, 50, 20, 10, 5, 4, 3, 2, 1 or 0.5. The surface area can be measured using standard ASTM C1274 or using N₂ adsorption with Brunauer-Emmett-Teller (BET) theory applied over the relative pressure range of 0.05 to 0.20 P/P₀ at 77 K.

The inert ceramic substrate layer may have a suitable density, for example to provide a degree of robustness. In some embodiments, the inert ceramic substrate layer has a density of between about 2.0 g/cm³ to about 15 g/cm³. The inert ceramic substrate layer may have a density (g/cm³) of at least about 2, 3, 4, 5, 6, 7, 8, 9, 10, 11, 12, 13, 14 or 15. The inert ceramic substrate layer may have a density (g/cm³) of less than about 15, 14, 13, 12, 11, 10, 9, 8, 7, 6, 5, 4, 3 or 2. The density may be a range provided by any two of these upper and/or lower values, for example between about 4 to 13 g/cm³.

The inert ceramic substrate layer has a suitable thickness. In some embodiments, the inert ceramic substrate layer has a thickness (in μm) of between about 0.01 to about 100,000, between about 0.1 to about 10,000, between about 1 to about 1000, or between about 1 to about 100, or between about 1 to about 10. The inert ceramic substrate layer may have a thickness (in μm) of at least about 0.001, 0.002, 0.005, 0.01, 0.015, 0.02, 0.05, 0.1, 0.5, 1, 5, 10, 50, 100, 200, 500, 700, 1000, 5000, 10,000 or 100,000. The inert ceramic substrate layer may have a thickness (in μm) of less than about 100,000, 10,000, 10,000, 5000, 1000, 700, 500, 200, 100, 50, 10, 5, 1, 0.5, 0.1, 0.05, 0.02, 0.015, 0.01, 0.005, 0.002, or 0.001. The thickness may be a range provided by any two of these upper and or lower values, for example between about 1 μm to about 1000 μm, between about 1 μm to about 500 μm, or between about 1 μm to about 100 μm. In one embodiment, the thickness of the metal oxide surface layer is less than the thickness of the underlying inert ceramic substrate layer.

The inert ceramic substrate layer may be formed from any suitable ceramic material. For example, the inert ceramic substrate comprises a suitable ceramic material which may have chemical affinity for the overlying metal oxide surface layer.

In some embodiments, the inert ceramic substrate layer may be selected from an inert oxide, an inert nitride, an inert carbide, an inert sulfide, an inert phosphate or a combination thereof. In some embodiments, the inert ceramic substrate layer is selected from quartz, a metal oxide, metal phosphate, metal nitride, metal carbide, metal sulfide, or combination thereof. The inert ceramic substrate layer may be selected from a metal oxide, metal phosphate, metal nitride, metal carbide, metal sulfide, or combination thereof. Other suitable ceramic materials include inert intermetallic compounds, for example a metal silicide, metal boride, or a metal selenide. In one embodiment, the inert ceramic substrate layer is a metal oxide (i.e. the inert ceramic substrate layer is a metal oxide substrate layer).

In one embodiment, the inert ceramic substrate layer is an inert oxide substrate layer, such as quartz, zirconia or tantalum oxide. In one embodiment, the inert ceramic substrate layer may be an oxide of silicon, tantalum, niobium, tungsten, molybdenum, vanadium, zirconium, titanium or aluminium, or mixed oxides thereof.

In one embodiment, the inert ceramic substrate layer is a metal oxide substrate layer. The metal oxide substrate layer may be an oxide of a valve metal, a refractory metal or another transition or main-block metal. The metal oxide substrate layer may be an oxide of tantalum, niobium, tungsten, molybdenum, vanadium, zirconium, titanium or aluminium, or mixed oxides thereof. The metal oxide substrate layer may be an oxide of tantalum, niobium, tungsten, molybdenum, vanadium, zirconium, or titanium, or mixed oxides thereof. In one embodiment, the inert ceramic substrate layer is quartz, tantalum oxide (Ta₂O₅) or zirconium oxide (ZrO₂). In one embodiment, inert ceramic substrate layer is tantalum oxide (Ta₂O₅) or zirconium oxide (ZrO₂), preferably tantalum oxide. Other metal oxides are also envisaged.

In one embodiment, the inert ceramic substrate layer is an inert oxide layer and a metal oxide surface layer is provided on the inert oxide layer. In one embodiment, the inert ceramic substrate layer is an inert oxide substrate layer, such as quartz, zirconia or tantalum oxide, and the metal oxide surface layer bound on the inert oxide substrate layer is an oxide of a valve metal, a refractory metal or another transition or main-block metal. In one embodiment, the inert ceramic substrate layer is an inert oxide substrate layer, such as quartz, zirconia or tantalum oxide, and the metal oxide surface layer bound on the inert oxide substrate layer is an oxide of a valve metal, a refractory metal or of tantalum, niobium, tungsten, molybdenum, vanadium, zirconium, or titanium, or a mixed oxide thereof. In one embodiment, the inert ceramic substrate layer is an inert oxide substrate layer, such as quartz, zirconia or tantalum oxide, and the metal oxide surface layer bound on the inert oxide substrate layer is an oxide of tantalum, niobium, tungsten, molybdenum, vanadium, zirconium, or titanium, or a mixed oxide thereof.

In one embodiment, the metal oxide surface layer is tantalum pentoxide (Ta₂O₅), which may be bound on the surface of the inert oxide substrate layer via the sol-gel process described herein. In one embodiment, the inert ceramic substrate layer is quartz, and the metal oxide surface layer bound on the quartz is an oxide of a valve metal, a refractory metal or of tantalum, niobium, tungsten, molybdenum, vanadium, zirconium, or titanium, or a mixed oxide thereof. In one embodiment, the inert ceramic substrate layer is tantalum oxide, and the metal oxide surface layer bound on the tantalum oxide is an oxide of a valve metal, a refractory metal or of tantalum, niobium, tungsten, molybdenum, vanadium, zirconium, or titanium, or a mixed oxide thereof.

In one embodiment, the inert ceramic substrate layer and metal oxide surface layer are each independently an oxide of a valve metal, a refractory metal or another transition or main-block metal. The inert ceramic substrate layer and metal oxide surface layer may each independently be an oxide of a valve metal, a refractory metal or of tantalum, niobium, tungsten, molybdenum, vanadium, zirconium, titanium or aluminium, or mixed oxides thereof. The inert ceramic substrate layer and metal oxide surface layer may each independently be an oxide of tantalum, niobium, tungsten, molybdenum, vanadium, zirconium, or titanium, or mixed oxides thereof. The inert ceramic substrate layer and metal oxide surface layer may each independently be tantalum oxide (Ta₂O₅) or zirconium oxide (ZrO₂), preferably tantalum oxide. Other metal oxides are also envisaged.

In a preferred embodiment, the inert ceramic substrate layer is a metal oxide substrate layer and comprises or consists of the same metal oxide as the overlying metal oxide surface layer described herein. In other words, the inert ceramic substrate layer and the metal oxide surface layer can be compositionally the same metal oxide. For example, both the metal oxide surface layer and inert ceramic substrate layer may comprise or consist of tantalum oxide or zirconium oxide.

In a related embodiment, the inert ceramic substrate layer and metal oxide surface layer together form a single metal oxide layer comprising parent radioisotope immobilised within the surface of the metal oxide layer as a radioisotope surface layer. Similar to the previous embodiment, in this case both the metal oxide surface layer and underlying inert ceramic substrate layer form the same metal oxide. When using the sol-gel process described herein according to some embodiments or examples, a gel formed using a metal alkoxide comprises branched metal-oxide-metal chains which can effectively adhere/graft to the pre-existing hydroxyl terminations naturally present on the surface of the underlying metal oxide substrate layer. As the gel dries, the colloidal network can evolve into an amorphous xerogel containing the parent radioisotope species, strongly bonded to the substrate. This ultimately converts into a metal oxide surface layer during the final heat treatment (e.g. calcination stage), effectively blending with the metal-oxide bonds of the underlying metal oxide substrate layer, being uniformly distributed across the entirety of the deposition area. So while each of the underlying metal oxide substrate layer and overlying metal oxide surface layer are prepared via different reactions (e.g. the substrate layer can be formed via oxidation e.g. thermal oxidation or anodisation of a metal substrate, and the surface layer formed via the sol-gel method described herein), the boundary between the metal oxide surface layer and underlying metal oxide substrate layer may be indistinguishable thus forms a single metal oxide layer (e.g. tantalum oxide underlayer and tantalum oxide surface layer, which are to most extents indistinguishable).

In one embodiment, there is provided a layered radioisotope source for generating a medically useful dose of daughter radioisotope through a chain of spontaneous decay from a parent radioisotope via a gaseous intermediate radioisotope, comprising:
an inert ceramic substrate layer;
a metal oxide surface layer bound on the inert ceramic substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert ceramic substrate layer;
a calcined metal oxide surface layer bound on the inert ceramic substrate layer; and
parent radioisotope immobilised on or within the calcined metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert ceramic substrate layer;
a tantalum oxide surface layer bond on the inert ceramic substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a inert ceramic substrate layer
a calcined tantalum oxide surface layer bound on the inert ceramic substrate layer; and
parent radioisotope immobilised on or within the calcined tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
an inert ceramic substrate layer; and
a tantalum oxide surface layer bound on the inert ceramic substrate layer; and ²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
an inert ceramic substrate layer;
a calcined tantalum oxide surface layer bound on the inert ceramic substrate layer; and ²²⁸Th immobilised on or within the calcined tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert ceramic substrate layer;
a sol-gel derived metal oxide surface layer bound on the inert ceramic substrate layer (e.g. the metal oxide surface layer is a sol-gel reaction product); and
parent radioisotope immobilised on or within the sol-gel derived metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert ceramic substrate layer;
a sol-gel derived tantalum oxide surface layer bond on the inert ceramic substrate layer (e.g. the tantalum oxide surface layer is a sol-gel reaction product); and
parent radioisotope immobilised on or within the sol-gel derived tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
an inert ceramic substrate layer; and
a sol-gel derived tantalum oxide surface layer bound on the inert ceramic substrate layer (e.g. the tantalum oxide surface layer is a sol-gel reaction product); and ²²⁸Th immobilised on or within the sol-gel derived tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert oxide substrate layer;
a metal oxide surface layer bound on the inert oxide substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert oxide substrate layer;
a calcined metal oxide surface layer bound on the inert oxide substrate layer; and
parent radioisotope immobilised on or within the calcined metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert oxide substrate layer;
a tantalum oxide surface layer bond on the inert oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a inert oxide substrate layer
a calcined tantalum oxide surface layer bound on the inert oxide substrate layer; and
parent radioisotope immobilised on or within the calcined tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
an inert oxide substrate layer; and
a tantalum oxide surface layer bound on the inert oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
an inert oxide substrate layer;
a calcined tantalum oxide surface layer bound on the inert oxide substrate layer; and
²²⁸Th immobilised on or within the calcined tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert oxide substrate layer;
a sol-gel derived metal oxide surface layer bound on the inert oxide substrate layer (e.g. the metal oxide surface layer is a sol-gel reaction product); and
parent radioisotope immobilised on or within the sol-gel derived metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
an inert oxide substrate layer;
a sol-gel derived tantalum oxide surface layer bond on the inert oxide substrate layer (e.g. the tantalum oxide surface layer is a sol-gel reaction product); and
parent radioisotope immobilised on or within the sol-gel derived tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a inert oxide substrate layer; and
a sol-gel derived tantalum oxide surface layer bound on the inert oxide substrate layer (e.g. the tantalum oxide surface layer is a sol-gel reaction product); and
²²⁸Th immobilised on or within the sol-gel derived tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal oxide substrate layer;
a metal oxide surface layer bound on the metal oxide substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal oxide substrate layer;
a calcined metal oxide surface layer bound on the metal oxide substrate layer; and
parent radioisotope immobilised on or within the calcined metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal oxide substrate layer;
a tantalum oxide surface layer bond on the metal oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal oxide substrate layer
a calcined tantalum oxide surface layer bound on the metal oxide substrate layer; and
parent radioisotope immobilised on or within the calcined tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal oxide substrate layer; and
a tantalum oxide surface layer bound on the metal oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal oxide substrate layer;
a calcined tantalum oxide surface layer bound on the metal oxide substrate layer; and
²²⁸Th immobilised on or within the calcined tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal oxide substrate layer;
a sol-gel derived metal oxide surface layer bound on the metal oxide substrate layer (e.g. the metal oxide surface layer is a sol-gel reaction product); and
parent radioisotope immobilised on or within the sol-gel derived metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal oxide substrate layer;
a sol-gel derived tantalum oxide surface layer bond on the metal oxide substrate layer (e.g. the tantalum oxide surface layer is a sol-gel reaction product); and
parent radioisotope immobilised on or within the sol-gel derived tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal oxide substrate layer; and
a sol-gel derived tantalum oxide surface layer bound on the metal oxide substrate layer (e.g. the tantalum oxide surface layer is a sol-gel reaction product); and
²²⁸Th immobilised on or within the sol-gel derived tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum oxide substrate layer;
a tantalum oxide surface layer bond on the tantalum oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum oxide substrate layer
a calcined tantalum oxide surface layer bound on the tantalum oxide substrate layer; and
parent radioisotope immobilised on or within the calcined tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum oxide substrate layer; and
a tantalum oxide surface layer bound on the tantalum oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum oxide substrate layer;
a calcined tantalum oxide surface layer bound on the tantalum oxide substrate layer; and
²²⁸Th immobilised on or within the calcined tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum oxide substrate layer;
a sol-gel derived tantalum oxide surface layer bound on the tantalum oxide substrate layer (e.g. the tantalum oxide surface layer is a sol-gel reaction product); and
parent radioisotope immobilised on or within the sol-gel derived tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum oxide substrate layer; and
a sol-gel derived tantalum oxide surface layer bound on the tantalum oxide substrate layer (e.g. the tantalum oxide surface layer is a sol-gel reaction product); and
²²⁸Th immobilised on or within the sol-gel derived tantalum oxide surface layer to allow for effective emanation of gaseous ²²⁰Rn away from the layered radioisotope source.

While not required, the inert ceramic substrate layer may be provided as a layer on a metal substrate. For example, the inert ceramic substrate layer may be bound to or derived from an underlying metal substrate. In one embodiment, the inert ceramic substrate layer may be an oxide bound to or derived from an underlying metal substrate. In another embodiment, the inert ceramic substrate layer is a metal oxide layer and is an oxide of the underlying metal substrate. For example, the metal substrate may be tantalum and the inert ceramic substrate layer may comprise or consist of tantalum oxide, which according to some embodiments or examples, may be formed by oxidising the tantalum metal substrate (e.g. via thermal oxidation or anodisation). The metal substrate may provide further advantages according to some embodiments or examples described herein, including radiation shielding, and in some cases the metal substrate can provide stable anchoring for the thinner inert ceramic substrate layer when in the source chamber described herein, for example, upon contact with a carrier gas.

Where a metal substrate is present, it will be appreciated that the inert ceramic substrate layer can be described as an intervening layer located between the metal oxide surface layer and the metal substrate.

The metal substrate may have a morphology suitable for inserting into a generator, for example into the source chamber described herein. In one embodiment, the metal substrate may be provided as a discrete unit, for example a disk, plate, film, platen, slab, tube, tube-section or monolith. The metal substrate may have any desired cross-sectional shape including, but not limited to spherical or semi-spherical. In one embodiment, the metal substrate is a disk or slab. The disk may be configured to be inserted into a radioisotope generator.

In some embodiments, the metal substrate has a thickness of between about 1 mm to about 100 mm. The metal substrate may have a thickness (in mm) of at least about 0.5, 1, 2, 3, 4, 5, 6, 7, 8, 9, 10, 12, 15, 20, 25, 30, 40, 50, or 100. The metal substrate may have a thickness (in mm) of less than about 100, 50, 40, 30, 25, 20, 15, 12, 10, 9, 8, 7, 6, 5, 4, 3, 2, 1 or 0.5. The thickness may be a range provided by any two of these upper or lower values, for example between about 1 mm to about 100 mm, for example between about 1 mm to about 100 mm, or between about 1 mm to about 20 mm, e.g. between about 1 mm to about 15 mm. The metal substrate may have a thickness effective to provide a flat surface for the inert ceramic substrate layer. In some embodiments, thicker metal substrates may be more amenable to recycling/reconditioning/reuse, for example rendering it safer and easier to strip unused radioisotope from the substrate. In one embodiment, the thickness of the inert ceramic substrate is less than the thickness of the underlying metal substrate.

In one embodiment, the surface of the metal substrate comprising the inert ceramic substrate layer has a surface area of between about 0.125 cm² to about 50 cm². The metal substrate comprising the inert ceramic substrate layer may have a surface area (in cm²) of at least about 0.125, 0.25, 0.5, 0.75, 1, 1.5, 2, 3, 4, 5, 10, 15, 20, 25, 30, 35, 40, 45 or 50. The metal substrate comprising the inert ceramic substrate layer may have a surface area (in cm²) of less than about 50, 45, 40, 35, 30, 25, 20, 15, 10, 5, 4, 3, 2, 1.5, 1, 0.75, 0.5, 0.25 or 1.25. A range may be provided by any two of these upper and/or lower values.

In one embodiment, the metal substrate is a refractory metal, a valve metal or other transition or main-block metal. The metal substrate may be tantalum, niobium, tungsten, hafnium molybdenum, vanadium, zirconium, titanium or aluminium, or alloys thereof. The metal substrate may be tantalum, niobium, tungsten, hafnium, molybdenum, vanadium, zirconium, or titanium, or alloys thereof. In one embodiment, the inert ceramic substrate is a metal oxide and is provided as a layer (e.g. surface layer) on a metal substrate or electrode thereof selected from tantalum, niobium, tungsten, molybdenum, vanadium, zirconium, titanium or aluminium, or alloys thereof.

In one embodiment, the metal substrate is an anodisable metal. The anodisable metal substrate or electrode thereof may be selected from tantalum, niobium, titanium or aluminium, or alloys thereof. The anodisable metal substrate or electrode thereof may be selected from tantalum, niobium, zirconium, titanium or aluminium, or alloys thereof.

In one embodiment, the metal of the metal substrate and the metal of the inert ceramic substrate layer (e.g. on the surface of the metal substrate) are the same. In one embodiment, the inert ceramic substrate layer is a metal oxide substrate layer, and the metal of the metal substrate and metal oxide substrate layer are the same. For example, the inert ceramic substrate layer may comprise or consist of a tantalum oxide layer and the metal substrate may comprise or consist of tantalum metal.

In one embodiment, where the inert ceramic substrate layer is a metal oxide, the metal oxide is produced by oxidatively pre-treating the surface of the metal substrate (e.g. by enhancing the native oxide layer on the surface of the metal substrate). The oxidation may also be induced by thermally oxidising the metal substrate in an oxygen atmosphere and/or by subjecting the metal substrate to more aggressive oxidising environments (e.g. an atmosphere with an elevated oxygen level).

The metal substrate has a suitable density. In some embodiments, the metal substrate has a density of between about 2 g/cm³ to about 20 g/cm³. The metal substrate may have a density (g/cm³) of at least about 1, 2, 5, 8, 10, 12, 15 or 20. The metal substrate may have a density (g/cm³) of less than about 20, 15, 12, 10, 8, 5, 4, 3 or 2. The density may be a range provided by any two of these upper and/or lower values, for example between about 5 to 20 g/cm³.

The metal substrate may have a roughened or textured surface. According to some embodiments or examples described herein, it was found that the roughened or textured surface of the metal substrate generated a textured metal oxide layer on the surface of the metal substrate, having an enhanced surface area and wettability which can enhance the coverage of the surface layer by a gel (e.g. an aerogel or xerogel) described herein to form a thin and uniform metal oxide surface layer comprising the immobilised parent radioisotope. It will be appreciated that such surface roughening or texturing is understood to mean that the surface of the metal substrate has been manipulated (i.e. roughened or textured) and does not encompass native “dead-flat” or polished metals which may have some form of microscopic roughness. In other words, the surface roughening is achieved by some physical or mechanical processing of the substrates surface, for example via abrading the surface using an abrasive powder (e.g. tungsten carbide) on an oscillating table, or by blasting the surface using a defined abrasive particles such as glass beads. The surface roughness may comprise angular patterns. In some cases, the roughened surface may have a peak count (Rpc) of less than 180 peaks/cm. The surface roughness may be measured using industry standard ASTM D7127, for example following abrasive treatment. In some embodiments, the roughened surface of the metal substrate has an increased % surface area compared to a corresponding non-roughened “dead-flat” substrate, for example at least about a 1, 2, 5, 10, 15 or 20% increase in surface area.

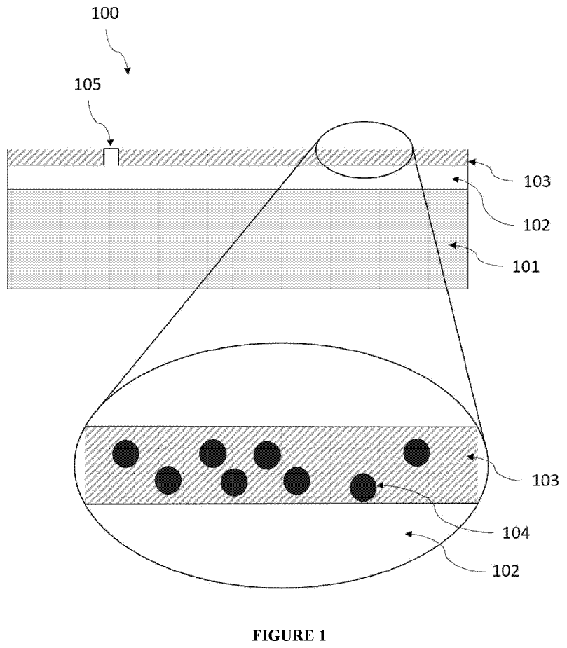

By way of example, a schematic of a radioisotope source immobilised parent radioisotope is provided in FIG. 1. The radioisotope source comprises a metal substrate (101) and an inert ceramic substrate layer (102) on the metal substrate. In one example, the inert ceramic substrate layer is a metal oxide layer. The metal oxide layer may be prepared by an oxidative treatment (e.g. via heating in air). A metal oxide surface layer is provided on the surface of the underlying metal oxide.

A metal oxide layer (103) is provided/formed on the surface of the inert ceramic substrate layer, and comprises parent radioisotope (104) immobilised on or within the metal oxide surface layer in a manner that allows for effective emanation of the gaseous intermediate radioisotope away from the layered radioisotope source.

It will be appreciated that the metal oxide surface layer does not have to be a continuous uniform layer covering the entire surface of the underlying inert ceramic substrate layer. For example, referring to FIG. 1, one or more small sections of the inert ceramic substrate layer (105) may protrude through the metal oxide surface layer (103) as result of non-uniform coverage during the sol-gel process used to prepare the radioisotope source. However, this is still considered to be a “surface layer” described herein, irrespective as to whether the metal oxide surface layer forms a continuous layer or forms one or more phases/sections decorating the surface of the inert ceramic substrate layer.

In one embodiment, there is provided a layered radioisotope source for generating a medically useful dose of daughter radioisotope through a chain of spontaneous decay from a parent radioisotope via a gaseous intermediate radioisotope, comprising:
a metal substrate;
an inert ceramic substrate layer provided on the metal substrate;
a metal oxide surface layer bound on the inert ceramic substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate;
an inert ceramic substrate layer provided on the metal substrate;
a calcined metal oxide surface layer bound on the inert ceramic substrate layer; and
parent radioisotope immobilised on or within the calcined metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate;
an inert oxide substrate layer (e.g. quartz, tantalum oxide or zirconia) provided on the metal substrate;
a sol-gel derived metal oxide surface layer bound on the inert ceramic substrate layer (e.g. the metal oxide surface layer is a sol-gel reaction product); and
parent radioisotope immobilised on or within the calcined metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate;
an inert oxide substrate layer (e.g. quartz, tantalum oxide or zirconia) provided on the metal substrate;
a metal oxide surface layer bound on the inert ceramic substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate;
an inert oxide substrate layer (e.g. quartz, tantalum oxide or zirconia) provided on the metal substrate;
a calcined metal oxide surface layer bound on the inert ceramic substrate layer; and
parent radioisotope immobilised on or within the calcined metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate;
an inert oxide substrate layer (e.g. quartz, tantalum oxide or zirconia) provided on the metal substrate;
a sol-gel derived metal oxide surface layer bound on the inert ceramic substrate layer (e.g. the metal oxide surface layer is a sol-gel reaction product); and
parent radioisotope immobilised on or within the calcined metal oxide surface layer to allow for effective emanation of the gaseous intermediate radioisotope away from the layered radioisotope source.

In one embodiment, the inert ceramic substrate layer is a metal oxide substrate layer, and the metal of the metal substrate and metal oxide substrate layer are the same. In one embodiment, the inert ceramic substrate layer is a metal oxide substrate layer which is formed by oxidatively pre-treating the surface of metal substrate. In one embodiment, the metal substrate is selected from the group consisting of tantalum, niobium, tungsten, hafnium, molybdenum, vanadium, zirconium, titanium or aluminium, or alloys thereof; and metal oxide layer is an oxide of the metal substrate. For example, the inert ceramic substrate layer may comprise or consist of a tantalum oxide layer and the metal substrate may comprise or consist of tantalum metal. In another example, the inert ceramic substrate layer may comprise or consist of a tantalum oxide layer which is produced by oxidatively pre-treating the surface of a tantalum metal substrate. In one embodiment, the metal oxide surface layer comprises or consists of the same metal oxide as the underlying metal oxide substrate layer. For example, the both the metal oxide surface layer and inert ceramic substrate layer may comprise or consist of tantalum oxide.

In one embodiment, the inert ceramic substrate layer is metal oxide substrate layer, and the metal of the metal substrate, metal oxide substrate layer and metal oxide surface layer is the same. In one embodiment, the inert ceramic substrate layer is an oxide of the metal substrate, and together with the metal oxide surface layer forms a single metal oxide layer on the metal substrate comprising the immobilised parent radioisotope.

In one embodiment, there is provided a layered radioisotope source for generating a medically useful dose of daughter radioisotope through a chain of spontaneous decay from a parent radioisotope via a gaseous intermediate radioisotope, comprising:
a metal substrate comprising on at least part of its surface a metal oxide substrate layer;
a metal oxide surface layer bound on the metal oxide substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal substrate comprising on at least part of its surface a metal oxide substrate layer;
a metal oxide surface layer bound on the metal oxide substrate layer; and
²²⁸Th immobilised on or within the metal oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal disk comprising on at least part of its surface a metal oxide substrate layer; and
a metal oxide surface layer bound on the metal oxide substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal disk comprising on at least part of its surface a metal oxide substrate layer; and
a metal oxide surface layer bound on the metal oxide substrate layer; and
²²⁸Th immobilised on or within the metal oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum metal substrate comprising on at least part of its surface a tantalum oxide substrate layer; and
a tantalum oxide surface layer bound on the tantalum oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum metal substrate comprising on at least part of its surface a tantalum oxide substrate layer; and
a tantalum oxide surface layer bound on the tantalum oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum metal disk comprising on at least part of its surface a tantalum oxide substrate layer; and
a tantalum oxide surface layer bound on the tantalum oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum metal disk comprising on at least part of its surface a tantalum oxide substrate layer; and
a tantalum oxide surface layer bound on the tantalum oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate comprising on at least part of its surface a metal oxide substrate layer;
a calcined metal oxide surface layer bound on the metal oxide substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal substrate comprising on at least part of its surface a metal oxide substrate layer;
a sol-gel derived metal oxide surface layer bound on the metal oxide substrate layer (e.g. the metal oxide surface layer is a sol-gel reaction product); and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal substrate comprising on at least part of its surface a metal oxide substrate layer;
a calcined metal oxide surface layer bound on the metal oxide substrate layer; and
²²⁸Th immobilised on or within the metal oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal substrate comprising on at least part of its surface a metal oxide substrate layer;
a sol-gel derived metal oxide surface layer bound on the metal oxide substrate layer (e.g. the metal oxide surface layer is a sol-gel reaction product); and
²²⁸Th immobilised on or within the metal oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal disk comprising on at least part of its surface a metal oxide substrate layer; and
a calcined metal oxide surface layer bound on the metal oxide substrate layer; and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a metal disk comprising on at least part of its surface a metal oxide substrate layer; and
a sol-gel derived metal oxide surface layer bound on the metal oxide substrate layer (e.g. the metal oxide surface layer is a sol-gel reaction product); and
parent radioisotope immobilised on or within the metal oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal disk comprising on at least part of its surface a metal oxide substrate layer; and
a calcined metal oxide surface layer bound on the metal oxide substrate layer; and
²²⁸Th immobilised on or within the metal oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a metal disk comprising on at least part of its surface a metal oxide substrate layer; and
a sol-gel derived metal oxide surface layer bound on the metal oxide substrate layer (e.g. the metal oxide surface layer is a sol-gel reaction product); and
²²⁸Th immobilised on or within the metal oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum metal substrate comprising on at least part of its surface a tantalum oxide substrate layer; and
a calcined tantalum oxide surface layer bound on the tantalum oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum metal substrate comprising on at least part of its surface a tantalum oxide substrate layer; and
a sol-gel derived tantalum oxide surface layer bound on the tantalum oxide substrate layer (e.g. the tantalum oxide surface layer is a sol-gel reaction product); and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum metal substrate comprising on at least part of its surface a tantalum oxide substrate layer; and
a calcined tantalum oxide surface layer bound on the tantalum oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum metal substrate comprising on at least part of its surface a tantalum oxide substrate layer; and
a sol-gel derived tantalum oxide surface layer bound on the tantalum oxide substrate layer (e.g. the tantalum oxide surface layer is a sol-gel reaction product); and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum metal disk comprising on at least part of its surface a tantalum oxide substrate layer; and
a calcined tantalum oxide surface layer bound on the tantalum oxide substrate layer; and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of daughter radioisotope through a chain of spontaneous decay from a parent radioisotope via a gaseous intermediate radioisotope, comprising:
a tantalum metal disk comprising on at least part of its surface a tantalum oxide substrate layer; and
a sol-gel derived tantalum oxide surface layer bound on the tantalum oxide substrate layer (e.g. the tantalum oxide surface layer is a sol-gel reaction product); and
parent radioisotope immobilised on or within the tantalum oxide surface layer to allow for effective emanation of the gaseous intermediate radioisotope away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum metal disk comprising on at least part of its surface a tantalum oxide substrate layer; and
a calcined tantalum oxide surface layer bound on the tantalum oxide substrate layer; and
²²⁸Th immobilised on or within the tantalum oxide surface layer to allow for effective emanation of gaseous ²²⁰Rn away from the layered radioisotope source.

In one embodiment, there is provided a layered radioisotope source for generating a medically useful dose of ²¹²Pb through a chain of spontaneous decay from ²²⁸Th radioisotope via gaseous ²²⁰Rn, comprising:
a tantalum metal disk comprising on at least part of its surface a tantalum oxide substrate layer; and
a sol-gel derived tantalum oxide surface layer bound on the tantalum oxide substrate layer (e.g. the tantalum oxide surface layer is a sol-gel reaction product); and
²²⁸Th immobilised on or within the tantalum oxide surface layer to allow for effective emanation of gaseous ²²⁰Rn away from the layered radioisotope source.

### Processes for Preparing the Layered Radioisotope Source

The present inventors have developed a sol-gel process for preparing the layered radioisotope source described herein. According to some embodiments or examples described herein, by controlling the reaction conditions and reagents of the sol-gel process, the thickness of the metal oxide surface layer and dispersion of immobilised parent radioisotope therein can be controlled resulting in a radioisotope source having improved properties, including for example lower parent radioisotope breakthrough due to the effective separation of gaseous intermediate radioisotope as it decays and emanates away from the immobilised parent radioisotope, and high resistance to radiolytic damage. These advantageous properties can lead to low levels of contamination of the parent radioisotope in the collected daughter radioisotope, and/or longer lifespan of the source, enabling the development of a radioisotope generator that allowed for “line-of-sight” gravity assisted collection of daughter radioisotope with minimal contamination.

In one embodiment, there is provided a sol-gel process for preparing a layered radioisotope source comprising: a) providing on the surface of an inert ceramic substrate layer a gel formed from a solution (e.g. sol) comprising a metal alkoxide and a parent radioisotope species: b) heating the gel under conditions effective to form a metal oxide surface layer bound on the inert ceramic substrate layer.

The radioisotope source, and in particular the metal oxide surface layer, can be prepared by a sol-gel process. The term “sol-gel” as used herein refers to the synthesis of solid materials from solution-state precursors, and involves the conversion of solubilised species into a colloidal solution (i.e. sol) that acts as the precursor for a network structure (i.e. gel), which is then heated to obtain the solid material.

By way of example only, the sol-gel technique can be generally summarized in the following steps, noting that some steps may be omitted and/or modified depending on the process conditions and/or reagents:
(i) Preparation of the ‘sol’ via the hydrolysis and partial condensation and commencement of gelling of dissolved precursor(s) in a suitable solvent—e.g. metal alkoxide precursors in an alcohol solution;
(ii) Further gelling via polycondensation to form more extensive metal-oxo-metal or metal-hydroxy-metal bonds.
(iii) and (iv): Aging and drying the gel where condensation continues within the gel network and solvent is removed to form a denser gel, e.g. a ‘xerogel’ via collapse of the porous network, an “aerogel” for example through supercritical drying, or a ‘cryogel’ for example through freeze-drying.
(v) Removal of surface water/hydroxy groups in M-OH units, through calcination at elevated temperature to form the ceramic material (e.g. a metal oxide).

A general overview of the sol-gel technique is outlined in Danks et al., Mater. Horiz., 2016, 3, 91.

The sol gel process for preparing the layered radioisotope source comprises: a) providing on the surface of an inert ceramic substrate layer a gel formed from a solution (e.g. sol) comprising a metal alkoxide and a parent radioisotope species; and b) heating the gel under conditions effective to form a metal oxide surface layer bound on the inert ceramic substrate layer, wherein the parent radioisotope is immobilised on or within the metal oxide surface layer to allow for effective emanation of the gaseous intermediate radioisotope away from the layered radioisotope source.

In one embodiment, the process described herein is for preparing a layered radioisotope source described herein under the section heading “Layered radioisotope sources”

### Oxidative Pre-Treatment of the Metal Substrate

The gel formed from a solution comprising a metal alkoxide and parent radioisotope species is provided on the surface of an inert ceramic substrate layer.

While the underlying inert ceramic substrate layer may comprise any suitable material that can support/affix to the metal oxide surface layer formed via the sol-gel process described herein, in one embodiment, the inert ceramic substrate layer may be a metal oxide layer provided by oxidatively pre-treating a metal substrate prior to providing (e.g. depositing) the gel thereon. In one embodiment, the process comprises the step of providing a metal oxide substrate layer which has been pre-prepared by oxidative pre-treatment of a metal substrate.

In one embodiment, the inert ceramic substrate is a metal oxide layer which is provided/formed on the surface of a metal substrate. The metal oxide layer may be prepared by surface modification of the metal substrate. In one embodiment, the metal oxide layer may be prepared by oxidising a metal substrate (i.e. a reaction in which electrons are removed) to prepare a metal oxide layer on the surface of the metal substrate. The oxidation of the surface of the metal substrate may be passive (i.e. occurring spontaneously in air) thereby forming a native metal oxide surface layer on the metal substrate, or it may be energetically driven by an oxidative pre-treatment of the metal surface. For example, the oxidation of the surface of the metal may comprise a chemical reaction with an oxidising agent to form the inert ceramic substrate as a surface oxide layer on the metal substrate.

In one embodiment, the metal substrate may be subjected to thermal oxidation (i.e. heating under an oxygen environment) to generate the metal oxide layer. Thermal oxidation is a well-understood process in which a metal (in its zero oxidation state) reacts with atmospheric oxygen at its surface to produce a definable layer of a metal oxide compound. The reaction relies on oxygen moving to the metal surface along cracks in pre-existing thin surface oxide films or by diffusion through such films. Oxygen diffusion is facilitated by temperature so heating the metal will increase both the reaction rate and the thickness of the metal oxide layer generated on the surface of the metal. The metal oxide layer may have variable stoichiometry and it may be initially formed as an amorphous material (with no defined lattice structure).

In one embodiment, the metal substrate is heated in the presence of oxygen to a temperature effective to form a layer of a metal oxide (e.g. the inert ceramic substrate) on the surface of the metal substrate. In one embodiment, the metal substrate is heated in the presence of oxygen to a temperature between about 100° C. to about 900° C. The metal substrate may be heated in the presence of oxygen at a temperature (in ° C.) of at least about 100, 200, 220, 240, 260, 280, 300, 320, 340, 360, 380, 400, 450, 500, 550, 600, 650, 700, 750, 800, 850 or 900. The metal substrate may be heated in the presence of oxygen at a temperature (in ° C.) of less than about 900, 850, 800, 750, 700, 650, 600, 550, 500, 450, 400, 380, 360, 340, 320, 300, 280, 260, 240, 220, 200 or 100. The heating temperature may be a range provided by any two of these upper and/or lower values, for example between about 200° C. to about 900° C., or between about 300° C. to about 800° C.

The metal substrate may be heated in the presence of oxygen from room temperature to the desired heating temperature at a rate of at least 1, 1.5, 2, 2.5, 2, 3.5, 3, 4, 4.5, 5, 6, 7, 8, 9 or 10° C./min. The heating rate may be a range provided by any two of these values.

The heating of the metal substrate may be a two-step heating process. In one embodiment, the metal substrate may be first heated in the presence of oxygen from an ambient temperature (e.g. room temperature) to a first temperature (in ° C.) of at least between about 100 to about 500. In one embodiment, the metal substrate may be first heated in the presence of oxygen from an ambient temperature (e.g. room temperature) to a first temperature (in ° C.) of at least about 100, 150, 200, 250, 300, 350, 400, or 500. In one embodiment, the metal substrate may be first heated in the presence of oxygen from an ambient temperature (e.g. room temperature) to a first temperature (in ° C.) of less than about 500, 400, 250, 300, 250, 200, 150 or 100. The first temperature may be range provided by any two of these upper and/or lower values, for example between about 200 to about 400.

In a further embodiment, the metal substrate may be further heated to a second temperature greater than the first temperature. In one embodiment, the metal substrate may be heated to a second temperature (in ° C.) of at least about 300, 350, 400, 450, 500, 550, 600, 650, 700, 750 or 800, provided that the second temperature is greater than the first temperature. The second temperature may be a range provided by any two of these values, for example between about 300 to about 900.

The metal substrate may be heated in the presence of oxygen for a period of time effective to form a layer of a metal oxide (e.g. the inert ceramic substrate) on the surface of the metal substrate. The metal substrate may be heated in the presence of oxygen for a period of time of between about 1 minutes to about 24 hours. The substrate may be heated in the presence of oxygen for a period of time of at least about 1, 5, 10, 15, 20, 25, 30, 35, 40, 45, 50, 55, 60, 65, 70, 75, 80, 85, or 90 (minutes), 2, 3, 4, 5, 6, 8, 10, 12, 14, 16, 18, 20, 22 or 24 hours. The metal substrate may be heated in the presence of oxygen for a period of time of less than about 24, 22, 20, 18, 16, 14, 12, 10, 8, 6, 5, 4, 3, 2 (hours), 90, 85, 80, 75, 70, 65, 60, 55, 50, 45, 40, 35, 30, 25, 20, 15, 10, 5, or 1 (minutes). The heating time may be a range provided by any two of these upper and/or lower values, for example between about 10 minutes to about 6 hours. Other heating temperatures and times to those recited herein are also envisaged, including longer heating times.

The metal substrate may be subjected to a surface roughening step to provide a textured surface prior to a thermal oxidation step. The surface roughening may be achieved by abrading the metal substrate with an abrasive material, for example alumina sandpaper.

### Formation of the Gel on the Surface of the Inert Ceramic Substrate

The sol-gel process comprises providing on the surface of an inert ceramic substrate layer a gel formed from a solution (e.g. sol) comprising a metal alkoxide and a parent radioisotope species.

The gel may be pre-formed in a suitable vessel and then deposited/coated onto the surface of the inert ceramic substrate (i.e. ex-situ formation and subsequent deposition, for example via dip-coating, pipetting or spin coating etc.), which is then subjected to heat treatment to form the metal oxide surface layer bound to the underlying inert ceramic substrate. Thus in one embodiment, step a) of the sol-gel process described herein comprises depositing a gel formed from a solution comprising a metal alkoxide and a parent radioisotope species on the surface of an inert ceramic substrate.

Alternatively, the gel may be formed on the surface of the inert ceramic substrate (i.e. in-situ formation). In one embodiment, step a) comprises depositing a solution comprising the metal alkoxide and parent radioisotope species on the surface of the inert ceramic substrate layer (e.g. by dip coating or pipetting etc.) to form the gel, which is then heat-treated to form the metal oxide surface layer bound to the underlying inert ceramic substrate.

The radioisotope species and metal alkoxide can be premixed to form the solution and/or gel which is then deposited onto the surface of the inert ceramic substrate. Alternatively, in one embodiment, step a) comprises preparing a first solution comprising the metal alkoxide and a second solution comprising the parent radioisotope species, and depositing the first solution and second solution sequentially, in any order, on the surface of the inert ceramic substrate layer to form the gel. The time period between depositing the first solution and second solution on the surface of the inert ceramic substrate layer can vary, but preferably is less than about 10 minutes, for example less than about 5 minutes. In one embodiment, the time period (in seconds) between depositing the first solution and second solution on the surface of the inert ceramic substrate layer is less than about 300, 240, 180, 120, 90, 60, 50, 40, 30, 20, 10 or 5. The time period may be a range provided by any two of these upper and/or lower values, for example between about 10 seconds to about 60 seconds.

It will be appreciated that over time, either spontaneously or shortly after mixing with the solution, the metal alkoxide will undergo hydrolysis/condensation to form the gel comprising a connected viscous and/or porous structure. The rate of hydrolysis/condensation can be controlled by the solvent used to prepare the solution (e.g. aqueous or non-aqueous/organic solvent). While not required, the solution and/or gel can be aged, which may further promote condensation and water loss within the gel network. This aging step may occur prior to deposition of the gel on the surface of the inert ceramic substrate (e.g. for ex-situ gel formation and deposition), or it may occur whilst the solution/gel is on the surface of the inert ceramic substrate (e.g. for in-situ gel formation). However it will be appreciated that this ageing step is different to the heating of the gel at step b) to obtain the metal oxide surface layer. Thus in one embodiment, prior to step b), the solution/gel is aged.

The aging of the gel may be for a period of time (in hours) of at least about 1, 2, 6, 12, 18, 24, 36, 48, 72, 96, 120, 144, 168, 192, 216, 240, 264, 288, 312, or 336 or more prior to the heating at step b). The gel may be aged for a period of time in a range provided by any two of these ageing times, for example between about 24 hours to 192 hours. The aging may be performed at ambient temperature or under low heating.

In one embodiment, the gel may be dried. Such drying can convert the viscous and/or porous gel network (which may be an aged gel described herein) into a denser amorphous thin gel layer on the surface of the inert ceramic substrate, e.g. in the form of a xerogel. In one embodiment, after step a) but prior to step b), the gel is dried, for example under conditions effective to form xerogel.

In one embodiment, the gel may be dried at a temperature (in ° C.) of between about 10 to about 180. The gel may be dried at a temperature (in ° C.) of at least about 10, 20, 30, 50, 100 or 150. The gel may be dried at a temperature (in ° C.) of less than about 180, 150, 100 or 50. The drying temperature may be a range provided by any two of these upper and/or lower values. However, it will be appreciated that the drying temperature may vary depending on the solvent used to prepare the solution comprising the metal alkoxide and parent radioisotope species. The length of the drying period can vary depending on the reagents, however in one embodiment, the gel can be dried for a period of time (in hours) of between about 0.1 to about 100, for example between about 1 to about 48.

In order for the sol-gel process to occur, it will be appreciated that the solution comprising the metal alkoxide and a parent radioisotope species contains an amount of water. The amount of water present can vary, but in some embodiments, is kept relatively low, which can provide further advantages such as a controlled/slower rate of hydrolysis/condensation. Furthermore, lower amounts of water in the solution can enhance the grafting of metal-oxide-metal chains/networks to pre-existing hydroxyl terminations that may be naturally present on the surface of the underlying inert ceramic substrate layer, which preferably is a metal oxide substrate layer.

In one embodiment, the water present in the solution is provided by the water of hydration of the parent radioisotope species, e.g. Th(NO₃)₄·5(H₂O). It will be appreciated that in this embodiment, no exogenous water has been added and the amount present in the solution/solvent is relatively low. In a related embodiment, the solution comprises between about 0.0001% w/w to about 15% w/w based on the total weight of the solution.

The solution used to prepare the gel at step a) may comprise any suitable solvent that can solubilise/suspend the metal alkoxide and parent radioisotope species. The solvent may be a single solvent or a mixture of solvents. The solvent may be water, an alcohol, an ester, a ketone, or an ether, including mixtures thereof.

In one embodiment, the solution at step a) comprises an alcoholic solution of the metal alkoxide and parent radioisotope species. In one embodiment, the alcoholic solution of the metal alkoxide and parent radioisotope species comprises an alcohol solvent in amount (in % v/v based on the total volume of the solution) of between about 50 to 99. According to some embodiments or examples described herein, the relative amount of an alcohol solvent (such as ethanol) may control the rate of hydrolysis of the metal alkoxide and suppress the formation of larger spherical clusters within the gel, resulting in a metal oxide surface layer having little to no ‘coffee-ring’-like deposition.

In one embodiment, the metal alkoxide is in stoichiometric excess relative to the parent radioisotope species (i.e. greater than 1 equivalents). In one embodiment, the molar ratio of metal alkoxide to parent radioisotope species is greater than 1, for example between greater than 1 and less than about 10. In one embodiment, the molar ratio of metal alkoxide to parent radioisotope species is greater than 1 and less than about 5, preferably greater than 1 and less than about 3. According to some embodiments or examples described herein, a slight excess of metal alkoxide e.g. 1.2-3 equivalents, can achieve fine dispersion of radioisotope within the metal oxide surface layer as well as good adhesion of the surface layer to the underlying inert ceramic substrate layer.

The metal alkoxide may be an alkoxide of a valve metal, a refractory metal or another transition or main-block metal. The metal alkoxide may be an alkoxide of tantalum, niobium, tungsten, molybdenum, vanadium, zirconium, titanium or aluminium, or mixed oxides thereof. The metal alkoxide may be an alkoxide of tantalum, niobium, tungsten, molybdenum, vanadium, zirconium, or titanium, or mixed oxides thereof. In one embodiment, the metal alkoxide is a tantalum alkoxide, e.g. tantalum ethoxide (e.g. Ta(OEt)₅).

In one embodiment, the inert ceramic substrate layer is a metal oxide substrate layer, and the metal of the metal alkoxide and underlying metal oxide substrate layer are the same. For example, the inert ceramic substrate layer may comprise or consist of a tantalum oxide layer and the metal alkoxide is a tantalum alkoxide (e.g. Ta(OEt)₅).

According to some embodiments or examples described herein, the gel formed using a metal alkoxide effectively traps the parent radioisotope species thus preventing significant migration and formation of larger discrete deposits of radioisotope, whilst the branched metal-oxide-metal chains of the gel can effectively adhere/graft to the pre-existing hydroxyl terminations naturally present on the surface of the underlying metal oxide substrate layer. As the gel dries, the colloidal network can evolve into an amorphous xerogel containing the parent radioisotope species, strongly bonded to the substrate. This ultimately converts into a metal oxide surface layer during the final heat treatment (e.g. calcination stage), effectively blending with the metal-oxide bonds of the underlying metal oxide substrate layer and thus creating very small radioisotope particles/phases embedded within the matrix of the metal oxide surface layer, being uniformly distributed across the entirety of the deposition area.

In one embodiment, the metal alkoxide is provided in the solution at a concentration of between about 0.00001 moles per litre (M) to about 1 M. In one embodiment, metal alkoxide is provided in the solution at a concentration (in M) of at least about 0.00001, 0.00005, 0.0001, 0.0005, 0.001, 0.005, 0.01, 0.02, 0.05, 0.08, 0.1, 0.2, 0.5, 0.8 or 1. In one embodiment, metal alkoxide is provided in the solution at a concentration (in M) of less than about 1, 0.8, 0.5, 0.2, 0.1, 0.08, 0.05. 0.02, 0.01, 0.005, 0.001, 0.0005, 0.0001, 0.00005 or 0.00001. The metal alkoxide concentration in the solution may be a range provided by any two of these upper and/or lower values, for example between about 0.00005 M and about 0.010 M.

The parent radioisotope species may be provided as a salt or hydrate thereof selected from one or more of hydroxides, halides, phosphates, nitrates, acetates, sulfates, perchlorates, ammonium compounds and anionic oxo-metallate compounds. In one embodiment, the parent radioisotope species is provided in solvated or complexed cationic form. In one embodiment, the parent radioisotope species is provided in an anionic oxo-metallate form (e.g. [ThO(HPO₄)₃(H₂PO₄)]⁵⁻).

In one embodiment, the parent radioisotope species may be a thorium compound or a radium compound, or a combination thereof. In one embodiment, the parent radioisotope species is a thorium species. The thorium radioisotope species may be selected from at least one of ²²⁷Th, ²²⁸Th and ²³²Th, or a combination thereof. In one embodiment, the parent radioisotope species is a thorium species provided as a nitrate salt or hydrate thereof, for example thorium nitrate (Th(NO₃)₄). In one embodiment, the parent radioisotope species is a radium species. The radium radioisotope species may be selected from at least one of ²²⁴Ra and ²²⁸Ra, or a combination thereof. In one embodiment, the parent radioisotope species is a radium species provided as a nitrate salt or hydrate thereof, for example radium nitrate. The radium radioisotope species may be provided as a hydrated cation thereof, for example, the hydrated radium divalent cation (Ra(H₂O)ₙ²⁺)

In one embodiment, the parent radioisotope species is provided in the solution at a concentration of between about 0.000001 M to about 1 M. In one embodiment, the parent radioisotope species is provided in the solution at a concentration (in M) of at least about 0.000001, 0.000005, 0.00001 0.00005, 0.0001, 0.0005, 0.001, 0.005, 0.01, 0.02, 0.05, 0.08, 0.1, 0.2, 0.5, 0.8 or 1. In one embodiment, the parent radioisotope species is provided in the solution at a concentration (in M) of less than about 1, 0.8, 0.5, 0.2, 0.1, 0.08, 0.05. 0.02, 0.01, 0.005, 0.001, 0.0005, 0.0001, 0.00005, 0.00001, 0.000005, or 0.000001. The parent radioisotope species concentration in the solution may be a range provided by any two of these upper and/or lower values, for example between about 0.00005 M and about 0.010 M.

The solution may further comprise an acid, for example nitric acid (HNO₃) or triflic acid (CF₃SO₃H), which can assist in the solubilisation and extraction of the parent radioisotope species into the solution. According to some embodiments or examples described herein, by acidifying the solution to a lower pH (i.e. more acidic pH) the soluble radioisotope species/ions remain well solvated and able to disperse throughout the gel without substantial aggregation across the surface of the inert ceramic substrate. In one embodiment, the acid is provided in the solution at a concentration of between about 0.1 M to about 3 M.

While not a requirement, the solution at step a) may further comprise one or more additional additives, such as binders, chelating agents, plasticizers etc., as understood by persons skilled in the art.

### Heating the Gel to Obtain the Metal Oxide Surface Layer Comprising Immobilised Parent Radioisotope

The gel is heated at step b) under conditions effective to form a metal oxide surface layer bound on the inert ceramic substrate. This heating step is essentially a thermal decomposition of the gel to burn-off and volatilise residual organic components and to dehydrate the gel to obtain the metal oxide surface layer. The heating at step b) may also convert the parent radioisotope species (e.g. de-nitration where nitrate ions are volatilised and expelled in the case of where the parent radioisotope species is provided as a nitrate salt) into discrete atoms and/or small oxide phases (e.g. less than 20 nm) which are uniformly distributed throughout the metal oxide surface layer, for example as a mixed oxide.

The gel at step b) may be heated to a suitable temperature effective to calcine the gel to form the metal oxide surface layer. In some embodiments, the gel is heated in step b) at a temperature (in ° C.) of between about 200 to 900. In some embodiments, the gel is heated in step b) at a temperature (in ° C.) of at least about 200, 250, 300, 350, 400, 450, 500, 550, 600, 650, 700, 750, 800, 850 or 900. In some embodiments, the gel is heated in step b) at a temperature (in ° C.) of less than about 900, 850, 800, 750, 700, 650, 600, 550, 500, 450, 400, 350, 300, or 250. The temperature may be a range provided by any two of these upper and/or lower values, for example between about 200 to about 500.

Any suitable heating rate, fast or slow, may be used, for example the gel may be heated from ambient temperature (e.g. room temperature) to the desired calcination temperature, for example the gel may be heated in step b) from ambient temperature a rate of between about 10° C./hour to about 250° C./hour.

The gel at step b) is heated for a period of time effective to calcine the gel to form the metal oxide surface layer. In some embodiments, the gel is heated in step b) for a period of time (in minutes) of between about 30 to about 360. In one embodiment, the gel is heated in step b) for a period of time (in minutes) of at least about 10, 15, 30, 45, 60, 90, 120, 150, 180, 210, 240, 270, 300, 330 or 360. In some embodiments, the gel is heated in step b) for a period of time (in minutes) of less than about 460, 330, 300, 270, 240, 210, 180, 150, 120, 90, 60, 45, 30, 15 or 10. The gel may be heated for a period of time in a range provided by any two of these upper and/or lower values, for example between about 100 minutes to about 350 minutes. Combinations of any one or more of the above temperatures and times are possible, for example, the gel may be heated in step b) at a temperature of between about 200° C. to 500° C. and for a period of time of between about 30 minutes to about 360 minutes.

According to some embodiments or examples described herein, calcining the gel at step b) at a low temperature of between about 200° C. to about 500° C. may provide one or more further advantages, including the formation of a metal oxide surface layer having a desirable degree of crystallinity while still being sufficient to decompose the initial gel/radioisotope species. An optimal degree of crystallinity can allow for better immobilisation of the parent radioisotope due to the surrounding atomic lattice being better able to self-heal following atomic displacements caused by energetic alpha-decay events. Additionally, an optimal degree of crystallinity of the metal oxide surface layer may also contribute to less parent radioisotope breakthrough and as a result longer radioisotope source lifetimes.

The heating (e.g. calcination) may be performed using a suitable furnace (e.g. muffle furnace), kiln, autoclave, microwave reactor, or hotplate.

### Radioisotope Generator

The present disclosure also provides a radioisotope generator for capturing a population of daughter radioisotope. In embodiment, there is provided a radioisotope generator defining a chamber for capturing a population of daughter radioisotope, the chamber configured to house a layered radioisotope source according to any aspects, embodiments or examples described herein. In embodiment, there is provided a radioisotope generator defining a chamber for capturing a population of daughter radioisotope, the chamber housing a layered radioisotope source according to any aspects, embodiments or examples described herein.

As described herein, the layered radioisotope source housed within the chamber may comprise parent radioisotope immobilised on or within the metal oxide surface layer in an amount effective to generate (i.e. produce) a medically useful dose of daughter radioisotope through a chain of spontaneous decay from the parent radioisotope via a gaseous intermediate radioisotope. It will therefore be understood that a chamber housing the layered radioisotope source both produces (i.e. generates) and captures a population of daughter radioisotope.

In one embodiment, the chamber comprises a collection surface and is configured to house the layered radioisotope source in the chamber, with the metal oxide surface layer facing (e.g. but not touching) the collection surface for collecting at least some of the emanated gaseous intermediate radioisotope for a period of time effective for it to decay into daughter radioisotope. In one embodiment, the chamber comprises a collection surface and houses the layered radioisotope source in the chamber, with the metal oxide surface layer facing (e.g. but not touching) the collection surface for collecting at least some of the emanated gaseous intermediate radioisotope for a period of time effective for it to decay into daughter radioisotope. In one embodiment, the emanated gaseous intermediate radioisotope may be collected for a period of time sufficient for it to accumulate into a medically useful amount of daughter radioisotope. For example, gaseous ²²⁰Rn may emanate away from the metal oxide surface layer and accumulate on the collection surface where it decays into ²¹²Pb.

The collection surface may be any surface capable of collecting and retaining emanated gaseous intermediate radioisotope, for example a removable dish/tray/container. The collection surface may comprise any suitable material. In one embodiment, the collection surface may comprise or consist of a cellulose material (such as cellulose filter paper), a polymeric material (e.g. PTFE), or glass. Alternatively, the collection surface may be the inner wall of a collection chamber as described herein.

In one embodiment, the chamber may be configured to house the layered radioisotope source wherein the metal oxide surface layer is in line-of-sight configuration with the collection surface. In one embodiment, the chamber houses layered radioisotope source wherein the metal oxide surface layer is in line-of-sight configuration with the collection surface. It will be understood that “line-of-sight” communication refers to a configuration where no obstacle (such as a closed valve or retractable seal) exists between the collection surface and metal oxide surface layer, thereby allowing for the efficient transport of emanated gaseous intermediate radioisotope. In some embodiments, the chamber may be configured with one or more valves, seals and/or closures configured to temporarily physically isolate/separate the layered radioisotope source from the collection surface, such as when the daughter radioisotope is extracted from the collection surface. While such physical separation of the layered radioisotope source from the collection surface temporarily disrupts the line-of-sight communication, it will be understood that when collection of emanated radioisotope is occurring, the chamber is configured at some point to provide line-of-sight communication between the metal oxide surface layer and the collection surface. In other words, such line-of-sight configuration does not preclude the presence of one or more closures, seals and/or valves being present in the chamber to temporarily physically isolate/separate the layered radioisotope source from the collection surface, such as when daughter radioisotope is being extracted from the collection surface.

In a related embodiment, the chamber may be configured to house the layered radioisotope source wherein the metal oxide surface layer substantially faces downwards to enable gravity assisted collection of at least some of the emanated gaseous intermediate on the collection surface. For example, the chamber may be configured to house the layered radioisotope source above the collection surface. Again, this does not preclude the presence of one or more closures, seals or valves being present in the chamber to physically isolate/separate the layered radioisotope source from the collection surface, such as when daughter radioisotope is being extracted from the collection surface. In another related embodiment, the chamber may house the layered radioisotope source wherein the metal oxide surface layer substantially faces downwards to enable gravity assisted collection of at least some of the emanated gaseous intermediate on the collection surface. For example, the chamber house the layered radioisotope source above the collection surface. Again, this does not preclude the presence of one or more closures, seals or valves being present in the chamber to physically isolate/separate the layered radioisotope source from the collection surface, such as when daughter radioisotope is being extracted from the collection surface.

In one embodiment, the radioisotope generator further comprises a carrier gas inlet port configured to introduce a carrier gas into the chamber to facilitate transfer of emanated gaseous intermediate radioisotope away from the layered radioisotope source onto the collection surface.

In one embodiment, the radioisotope generator further comprises a vacuum pump configured to apply a vacuum and evacuate the chamber to facilitate transfer of emanated gaseous intermediate radioisotope away from the layered radioisotope source onto the collection surface.

In one embodiment, the radioisotope generator further comprises a fluid delivery system configured to introduce a collection fluid into the chamber to collect daughter radioisotope from the collection surface. In some embodiments, the radioisotope generator comprises one or more valves, seals and/or closures configured to physically isolate/separate the layered radioisotope source from the collection surface within the chamber, such as when the daughter radioisotope is extracted from the collection surface. In a related embodiment, the radioisotope generator further comprises a collection fluid outlet port configured to transfer the collection fluid comprising daughter radioisotope from the chamber.

The radioisotope generator may further comprise a system for washing daughter radioisotope product from the collection surface into a product vessel, using an appropriate collection fluid. For example, the generator may further comprise a fluid delivery system configured to introduce a collection fluid into the chamber to collect daughter radioisotope which has deposited on the collection surface. The fluid delivery system may comprise a collection fluid reservoir coupled to a collection fluid inlet port via a collection fluid inlet valve for introducing the collection fluid into the chamber. The fluid delivery system may be controlled by a collection fluid inlet valve operably configured to intermittently open to introduce a series of pulses of the collection fluid through the collection fluid inlet port into the chamber.

The generator may further comprise a collection fluid outlet port configured to transfer the collection fluid comprising the daughter radioisotope from the chamber, for example into a product vessel. The collection fluid outlet port may be controlled by a collection fluid outlet valve and/or pump operably configured to intermittently open to extract the collection fluid comprising the daughter radioisotope from the chamber.

In one embodiment, the generator, in use, is configured to produce at least one medical (e.g. clinical or pre-clinical) dose of daughter radioisotope within a 24 hour period, for example at least 1, 2, 3, 4 or 5 medical doses of daughter radioisotope within a 24 hour period.

In one embodiment, the generator, in use, may be configured to produce at least one medical dose of daughter radioisotope (e.g. ²¹²Pb) of at least about 1, 2, 5, 10, 50, 60, 90, 120, 150, 200, 250, 300, 400, 500, 600, 700, 800, 900 or 1,000 MBq. In another embodiment, the generator, in use, may be configured to produce at least one medical dose of daughter radioisotope (e.g. ²¹²Pb) of less than about 1,000, 900, 800, 700, 600, 500, 400, 300, 250, 200, 150, 120, 90, 60, 50, 10, 5, 2 or 1. In one embodiment, the generator, in use, may be configured to produce at least one medical dose of daughter radioisotope (e.g. ²¹²Pb) of at least about 50, 70, 100, 120, 140, 160, 180 or 200 MBq. The generator, in use, may be configured to produce at least one medical dose of daughter radioisotope (e.g. ²¹²Pb) in a range provided by any two of these upper and/or lower values, for example between about 50 MBq to about 200 MBq.

It will be appreciated that any aspects, embodiments or examples of the process for producing and capturing daughter radioisotope and/or layered radioisotope source as described herein may form one or more aspects, embodiments or examples of the generator.

### Systems

The present disclosure also provides a system for producing and capturing a population of daughter radioisotope generated through a chain of spontaneous decay from a parent radioisotope, the system comprising: a) a generator; and b) a layered radioisotope source according to any aspect, embodiment or examples described herein.

In one embodiment, there is provided a system for producing and capturing a population of daughter radioisotope generated through a chain of spontaneous decay from a parent radioisotope, the system comprising: a) a radioisotope generator defining a chamber for producing and capturing a population of daughter radioisotope; and b) a layered radioisotope source according to any aspect, embodiment or examples described herein housed in the chamber.

The radioisotope generator may be a generator according to any aspects, embodiments, or examples described herein.

It will be appreciated that any aspects, embodiments or examples of the radioisotope generator, process for producing and capturing daughter radioisotope, and/or layered radioisotope source as described herein may form one or more aspects, embodiments or examples of the system.

### Process for Producing and Capturing Daughter Radioisotope

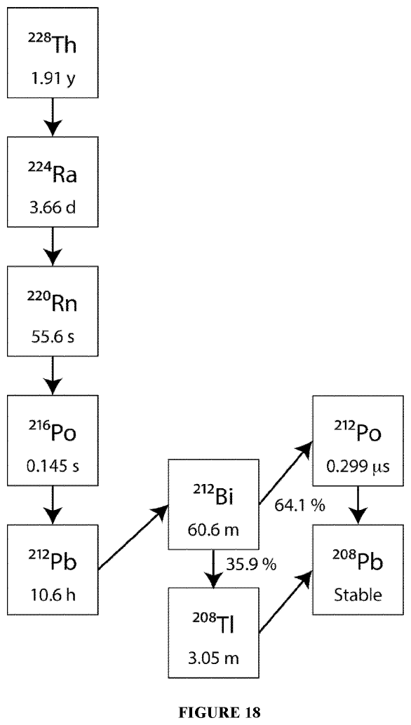

The present disclosure also relates to processes for capturing a population of daughter radioisotope. The daughter radioisotope produced by the process described herein is generated through a chain of spontaneous decay from a parent radioisotope via a gaseous intermediate radioisotope. An example of a spontaneous radioactive decay series relating to the present disclosure is provided in FIG. 18, illustrating the ²²⁸Th decay series.

In one embodiment, there is provided a process for capturing a population of daughter radioisotope comprising: a) allowing for the emanation of a gaseous intermediate radioisotope generated through a chain of spontaneous decay from a parent radioisotope immobilised on or within a layered radioisotope source according to any aspect, examples or embodiments described herein; and b) collecting at least some of the gaseous intermediate radioisotope for a period of time effective for it to decay into daughter radioisotope. In one embodiment, emanated gaseous intermediate radioisotope may be collected for a period of time sufficient for it to accumulate into a useful amount of daughter radioisotope.

The process may comprise a radioisotope generator according to any aspects, embodiments or examples described herein or a system according to any aspects, embodiments or examples described herein.

In one embodiment, the parent radioisotope is an alpha-emitting radioisotope. In one embodiment, the parent radioisotope is a thorium radioisotope selected from at least one of thorium-227 (²²⁷Th) and thorium-228 (228Th). In one embodiment, the gaseous intermediate radioisotope is a radon radioisotope selected from at least one of radon-219 (²¹⁹Rn) and radon-220 (²²⁰Rn). In one embodiment, the daughter radioisotope is a lead radioisotope selected from at least one of lead-211 (²¹¹Pb) or lead-212 (²¹²Pb).

In one embodiment, the process further comprises a step of recovering at least some of the daughter radioisotope. The recovered daughter radioisotope can be used in a radiopharmacy. For example, the recovered daughter radioisotope may be conjugated to a targeting molecule, such as a cancer cell targeting molecule, including for use as a radiopharmaceutical agent such as in radioligand therapy. Various applications and uses of the recovered daughter radioisotope are described herein, including under the heading “Applications” in the present disclosure.

It will be appreciated that any aspects, embodiments or examples of the radioisotope generator, system, and/or layered radioisotope source as described herein may form one or more aspects, embodiments or examples of the process for producing and capturing daughter radioisotope.

### Applications

The applications of the present disclosure include various applications in the medical, therapeutic and diagnostic fields, including for example, as radiopharmaceutical agents for treating cancer. For example, the nuclear medicine field provides for the radiolabelling of macromolecules such as antibodies that bind with high specificity to antigens expressed on particular cancer cells. Alpha-particle emitters (including daughter radioisotope generated, captured and collected as described herein) are particularly effective as short range cytotoxic payloads on such targeted molecular vehicles, thereby allowing for cancer cell destruction with minimal impact on surrounding healthy tissue.

In some embodiments, the daughter radioisotope (e.g. ²¹²Pb) produced using the layered radioisotope source, generator, system and process described herein are well suited for being attached to cancer targeting molecules since it is of high radiochemical purity and of high specific activity (for example owing to the low ²⁰⁸Pb content as a result of using the generator and process described herein). The generation of high purity daughter radioisotope (e.g. high purity ²¹²Pb) using the generator and process described herein will allow for the production of this therapeutic isotope at scale with wide geographic distribution.

The daughter radioisotope, for example ²¹²Pb generated using the layered radioisotope source, process, generator and system described herein can be directly utilised in various clinical applications, including conjugation to targeting molecules/ligands for use in radiopharmacy applications, such as radioligand therapy. Examples of targeting molecules/ligands include antibodies and/or peptides, for example prostate-specific membrane antigen (PSMA) ligands. The targeting molecule/ligand may include that described in International PCT Application No. PCT/AU/2023/050763 filed on 11 Aug. 2023, the contents of which are incorporated herein by reference in their entirety.

The present application claims priority from Australian Provisional Patent Application No. 2023900421 filed on 20 Feb. 2023, the contents of which are incorporated herein by reference in their entirety.

### EXAMPLES

In order that the disclosure may be more clearly understood, particular embodiments of the invention are described in further detail below by reference to the following non-limiting experimental materials, methodologies and examples.

### Example 1: Sol-Gel Process for Preparing Layered Radioisotope Source

### Preparation of Inert Ceramic Substrate Surface Layer by Thermal Oxidation

An inert ceramic substrate layer composed of tantalum pentoxide (Ta₂O₅) was formed on a tantalum metal disk. Briefly, the tantalum disk was placed on a sheet of 240 grit alumina (Al₂O₃) sandpaper on a clean, smooth surface, and one face of the tantalum disk was abraded until a uniform matt finish was obtained. The disk surface was then washed with a detergent solution, and then rinsed with DI water.

The washed and dried abraded tantalum disk was placed in a furnace and heated according to the following temperature-time profile: heat from ambient temperature (e.g. room temperature) to 300° C. over 60 minutes; increase furnace temperature from 300° C. to 575° C. over 40 minutes; hold at 575° C. for 45 minutes; commence slow natural cooling to ambient temperature (ambient cooling).

Similarly, an inert ceramic substrate layer composed of zirconia (ZrO₂) was formed on a zirconium metal disk using the above protocol, but using the following furnace heating program: heat from ambient temperature (e.g. room temperature) to 750° C. over 120 minutes, hold at 750° C. for 300 minutes, allow slow natural cooling to ambient temperature.

### Extraction of Parent Radioisotope Species

To prepare a stock solution of thorium nitrate, 200 μL of EtOH was deposited into a V-vial containing a small amount of ²²⁸Th(NO₃)₄ pentahydrate. The solution was then transferred to an empty 1.5 mL Eppendorf tube and the aforementioned steps repeated a further 2 times to give a total volume of 600 μL of ethanolic ²²⁸Th(NO₃)₄ solution. The Eppendorf tube was then placed in heating block set to 70° C. and heated until the total volume of the ethanolic ²²⁸Th(NO₃)₄ solution was reduced to 200 μL.

In some instances, nitric acid (HNO₃) was added to aid in extraction of the ²²⁸Th(NO₃)₄, such that a final solution of 4.3 mM ²²⁸Th(NO₃)₄ and 0.4 mM HNO₃ was obtained. 58 μL of this solution was then transferred to a 1.5 mL Eppendorf tube and the solution was slowly evaporated on a hot plate until approximately 5-10 μL was left in the tube.

### Preparation of Metal Alkoxide Stock Solution

A 0.128 M stock solution of Ta(OEt)₅ in ethanol was prepared by adding 500 μL of neat Ta(OEt)₅ to 14.5 mL of EtOH.

Deposition of Metal Alkoxide Solution/Gel Comprising Parent Radioisotope Species onto Metal Oxide Surface Layer

Typically, a predetermined amount of Ta(OEt)₅ and ²²⁸Th(NO₃)₄ stock solutions prepared above were mixed, optionally diluted with either H₂O and/or EtOH. Optionally, the Ta/²²⁸Th mixture was allowed to age in a container for a period of up to 7 days prior to deposition. The mixture was then deposited at the centre of the roughened face of the prepared disks comprising either the Ta₂O₅ surface or ZrO₂ surface. The disk was then dried under ambient conditions overnight to allow the sol-gel process to complete forming a gel layer (e.g. xerogel) on the metal oxide surface of the disk.

Sequential Deposition of Metal Alkoxide and Parent Radioisotope Species onto Metal Oxide Surface Layer

Typically, separate solutions comprising a predetermined amount of Ta(OEt)₅ stock solution and ²²⁸Th(NO₃)₄ solution prepared above were each diluted with water and/or EtOH, and then sequentially deposited at the centre of the roughened face of the prepared disks comprising either the Ta₂O₅ surface or ZrO₂ surface. The disk was then dried under ambient conditions overnight to allow the sol-gel process to complete forming a gel layer (e.g. xerogel) on the metal oxide surface of the disk.

### Calcination/De-Nitration of the Gel to Form the Metal Oxide Surface Layer Comprising Immobilised Parent Radioisotope

The xerogel coated disk was placed in a furnace heated according to the following program to give the final TaₓTh_{y}O_{z} coated tantalum or zirconia disk, respectively: a) heat from ambient temperature to 280° C. over 150 min, b) hold temperature at 280° C. for 150 minutes; c) commence slow natural cooling to ambient temperature (ambient cooling).

In order to optimize the properties of the final TaₓTh_{y}O_{z} mixed oxide surfaces, a number of layered radioisotope sources were prepared in line with the above protocol, varying the molar ratio of both the tantalum and thorium species, mode of deposition, solvent and aging times.

Table 1 provides a summary of the sol-gel process conditions investigated using the above general protocols.

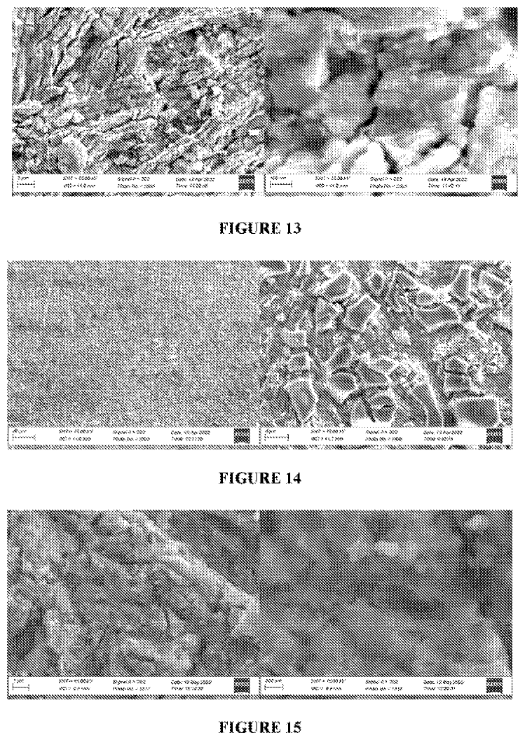

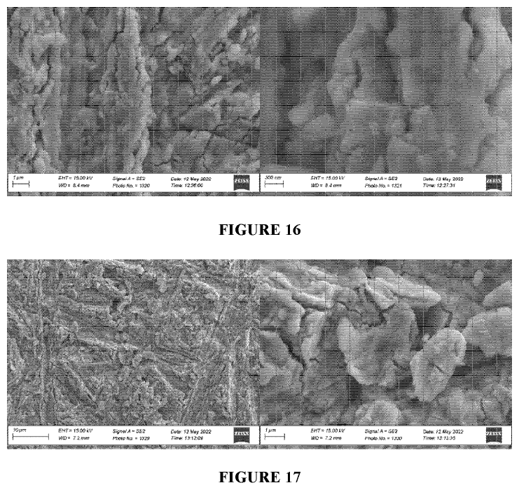

TABLE 1
Trialed film deposition conditions and resultant film properties.

| Sample | n(Th) | n(Ta) | | | |
|---|---|---|---|---|---|
| ID | (nmol) | (nmol) | Deposition steps | Visual aspect | SEM morphology |
| NC018T | 511.7 | 3200 | Pre-mixed Ta solution, Th | Light ‘coffee | Small ‘dried mud’-like |
| | | | solution, 356 μL H₂O in | ring’ visible, | deposits around deposition |
| | | | Eppendorf tube (500 μL total | very minor | edge, comprised of sub |
| | | | volume), degassed solution, | flaking around | 100 nm particles, containing |
| | | | then deposited on the Ta₂O₅ | ring, uniform | both Ta and Th (FIGS. 9, |
| | | | surface of the Ta disk. | ‘matte grey’ | edge region, and 10, |
| | | | Dried then calcined. | inside ring. | bulk region). |
| NC023T | 511.7 | 3200 | Pre-mixed Ta solution, Th | Shaded regions, | Some cracked film'- |
| | | | solution, 356 μL H₂O 250 | minor flaking | like deposits, |
| | | | μL EtOH in Eppendorf tube | around region | containing both Ta |
| | | | (750 μL total volume), | where it dried last | and Th (FIG. 11, |
| | | | degassed solution, then | (bubbles were | edge region). |
| | | | deposited on the Ta₂O₅ | present), faint | |
| | | | surface of the Ta disk. | ‘coffee ring’. | |
| | | | Dried then calcined. | | |
| NC024T | 511.7 | 640 | Pre-mixed 10 μL Ta solution, | Very light ‘coffee | Very small (<50 nm) bright |
| | | | 248 μL Th solution (after | ring’ visible, | dots (high Th), more |
| | | | dilution), 742 μL H₂O, in | uniform ‘matte | concentrated in some areas |
| | | | Eppendorf tube (1 mL total | grey’ inside ring. | (FIGS. 12, edge region, |
| | | | volume), degassed solution, | | and 13, bulk region). |
| | | | then 479 μL of solution | | |
| | | | deposited on the Ta₂O₅ | | |
| | | | surface of the Ta disk. Dried | | |
| | | | then calcined. | | |
| NC025T | 511.7 | 15232 | Pre-mixed Ta(OEt)₅ solution, | ‘Coffee ring’ | Some thick, ‘dried mud’-like |
| | | | Th solution, 356 μL H₂O in | visible, flaking | deposits with poor adhesion |
| | | | Eppendorf tube (594 μL total | around ring and | (FIG. 14, bulk region). |
| | | | volume), degassed solution, | central area. | |
| | | | then deposited on the Ta₂O₅ | | |
| | | | surface of the Ta disk. Dried | | |
| | | | then calcined. | | |
| NC026T | 197.8 | 200 | Pre-mixed 10 μL Ta(OEt)₅ | No ‘coffee ring’ | Very thin ‘cracked film’-like |
| | | | solution and 301 μL Th | visible, uniform | deposits (FIGS. 15, edge |
| | | | solution in Eppendorf tube, | distribution | region, and 16, bulk region). |
| | | | pre-wet disk with 100 μL | (underlying Ta₂O₅ | |
| | | | water, tilt-spread to extend | pattern blurred). | |
| | | | to entire surface, then 49 μL | ‘Bleed-in’ from | |
| | | | of solution deposited on | PAP pen? | |
| | | | the Ta₂O₅ surface of the | | |
| | | | Ta disk. Dried then calcined. | | |
| NC027T | 250 | 260 | Pre-mixed 10 μL Ta(OEt)₅ | No ‘coffee ring’ | Very thin ‘cracked film’-like |
| | | | solution, 66 μL Th solution, | visible, uniform | deposits (FIG. 17, bulk |
| | | | 101 μL EtOH, 33 μL HNO₃ | distribution | region). |
| | | | 70% (final conc. 2.5 M) in | (underlying Ta205 | |
| | | | Eppendorf tube and then 40 | pattern blurred). | |
| | | | μL of solution deposited on | | |
| | | | the Ta₂O₅ surface of the Ta | | |
| | | | disk. Dried then calcined. | | |
| NC028T | 250 | 260 | Pre-mixed 10 μL Ta(OEt)₅ | No ‘coffee ring’ | Very uniform dispersed Th |
| | | | solution, 66 μL Th solution, | visible, uniform | signal, ‘thin film’. Some |
| | | | 134 μL EtOH in Eppendorf | distribution | bright dots near edge |
| | | | tube, then 40 μL of solution | (underlying Ta₂O₅ | (FIG. 2). |
| | | | deposited on the Ta₂O₅ | pattern blurred). | |
| | | | surface of the Ta disk. Dried | | |
| | | | then calcined. | | |
| NC030T | 249.4 | 256 | Deposited on the Ta₂O₅ | No ‘coffee ring’ | Some ‘dried mud’-like |
| | | | surface of the Ta disk. (in | visible, uniform | deposits around deposition |
| | | | this order): 75 μL H₂O, 267 | distribution | edge. |
| | | | μL HNO₃, 58 μL Th stock | (underlying Ta₂O₅ | |
| | | | solution, 20 μL Ta(OEt)₅ | pattern blurred). | |
| | | | solution (after dilution). Dried | | |
| | | | then calcined. | | |
| NC036T | 240 | 170 | Pre-diluted Ta(OEt)₅ and Th | Some drying | Some ‘fuzzy’ deposits near |
| | | | stock solutions in separate | marks present | deposition edge, very weak |
| | | | eppendorf tubes with EtOH to | before de-nitration | Th signal present in some |
| | | | reach 100 μL and 150 μL | but disappeared | bulk spectra. |
| | | | volumes, respectively. | after process. | |
| | | | Deposited Ta solution on to | Uniform | |
| | | | the Ta₂O₅ surface of the Ta | distribution- no | |
| | | | disk., then Th solution | flaking. | |
| | | | deposited immediately after | | |
| | | | (total volume 250 μL). Dried | | |
| | | | then calcined. | | |
| NC037T | 240 | 360 | Pre-diluted Ta(OEt)₅ and Th | No ‘coffee ring’ | Some ‘dried mud’ deposits |
| | | | stock solutions in separate | visible, uniform | near edges, with round |
| | | | Eppendorf tubes with EtOH to | distribution | holes. |
| | | | reach 100 μL and 150 μL | (underlying Ta₂O₅ | |
| | | | volumes, respectively. Added | pattern blurred). | |
| | | | Ta solution first, then Th | No flaking. | |
| | | | solution immediately after | | |
| | | | (total volume 250 μL). Dried | | |
| | | | then calcined. | | |
| NC043T | 250 | 260 | Pre-mixed 10 μL Ta(OEt)₅ | Slightly poorer | Mostly uniform film (with |
| | | | solution, 66 μL Th solution, | wetting compared | small cracks) with equal |
| | | | 134 μL EtOH in Eppendorf | to Ta₂O₅ surfaces | Ta/Th atomic ratios (1-1.2) |
| | | | tube, then 40 μL of solution | leading to reduced | throughout it. Thickness of |
| | | | deposited on ZrO₂ surface of | deposition area; | film slightly variable, |
| | | | Ta disk. Dried then calcined. | | larger/more cracks around |
| | | | | | deposition edges (FIGS. 5 |
| | | | | | and 6). |
| NC046T | 260 | 240 | Pre-mixed 10 μL Ta(OEt)₅ | No ‘coffee ring’ | Before calcination/de- |
| | | | solution, 66 μL Th solution, | visible, uniform | nitration: no discernible |
| | | | 134 μL EtOH in Eppendorf | distribution | deposits; but Th signal |
| | | | tube, left for 7 days. Then 40 | (underlying Ta₂O₅ | present in all sampled |
| | | | μL of solution deposited on | pattern blurred). | regions. After |
| | | | the Ta₂O₅ surface of the Ta | | calcination/de-nitration: no |
| | | | disk. Dried then calcined. | | obvious difference (FIG. |
| | | | | | 4). |
| NC050T | 250 | 260 | On smoother (polished to | No ‘coffee ring’ | Very thin ‘cracked film’-like |
| | | | 2000 grit before baking) | visible, uniform | deposits around edges. Th |
| | | | Ta₂O₅ surface: transferred 58 | distribution | signal present in bulk. |
| | | | μL Ta stock solution into | (underlying Ta₂O₅ | |
| | | | Eppendorf, heated on hotplate | pattern blurred, | |
| | | | until tiny droplet remains, | possibly thicker in | |
| | | | then added 140 μL Ta | some areas). No | |
| | | | solution, deposited all on | flaking. | |
| | | | the smooth oxide surface. | | |
| | | | Left to dry for 5 hours | | |
| | | | before calcination. | | |
| NC051T | 250 | 260 | Transferred 58 μL Ta stock | No ‘coffee ring’ | Very thin ‘cracked film’-like |
| | | | solution in EtOH/aqueous | visible, uniform | deposits around edges. Th |
| | | | HNO₃ into an Eppendorf, | distribution | signal present in bulk. |
| | | | heated on hotplate until tiny | (underlying Ta₂O₅ | |
| | | | droplet remains, then added | pattern blurred). | |
| | | | 140 μL Ta solution, deposited | No flaking. | |
| | | | all onto Ta₂O₅ surface of Ta | | |
| | | | disk. Left to dry for 5 hours | | |
| | | | before calcination. | | |

### Example 2: Morphological and Compositional Analysis of the Deposited TaₓTh_{y}O_{z} Films

The developed sol-gel protocol for the most part produced a surface morphology in which the thorium is present in the form of a ‘thin film’ of homogenously mixed tantalum and thorium oxide.

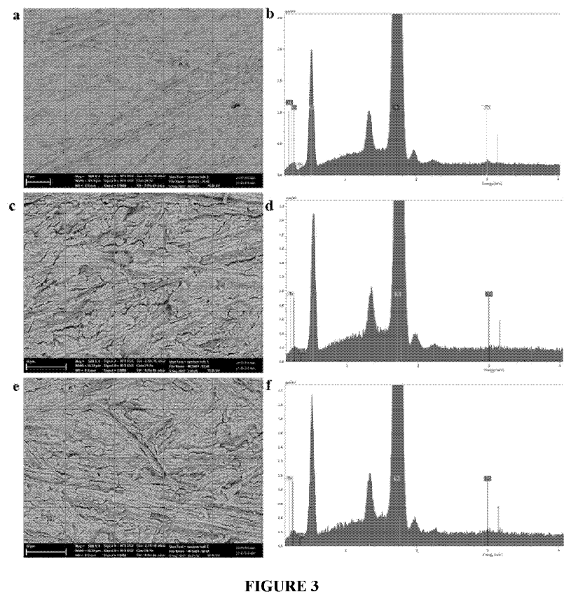

SEM inspection of samples NC028T and NC046T revealed no discrete particles of thorium oxide or thorium-rich features down to a size of about 20 nm or less (see FIGS. 2a, 2c, 3a, 3,c and 3e, NC828T and NC046T, respectively). The lack of discrete thorium deposits of this size suggests that thorium atoms/species are smoothly distributed and uniformly mixed within a deposited amorphous or semi-crystalline TaₓTh_{y}O_{z} films.

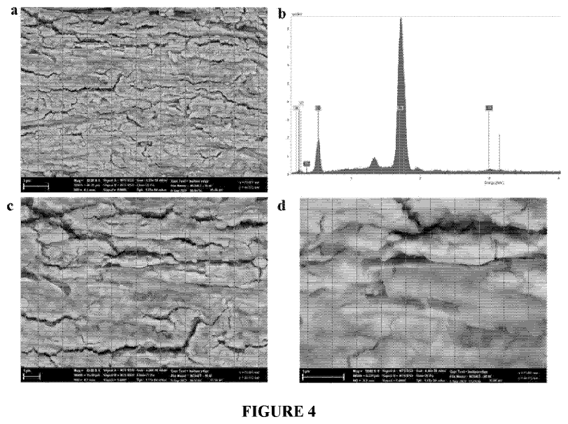

EDX analysis of samples NC028T and NC046T showed a signal at approximately 3 keV, corresponding to the thorium M line, across the surface of the disk, indicating that the deposited TaₓTh_{y}O_{z} film was present in all of the acquired SEM images of the samples (see FIGS. 2b, 2d, 3b, 3d and 3f). In areas near the edge of the disks, the intensity of the thorium M line signal was greater, due to the thickness of the deposited film being slightly thicker toward the edges of the disk (see FIG. 4b, NC046T). Even in these thicker film regions, the intensity of the thorium M line signal stayed below 0.65%, suggesting the presence of thorium only in atomically thin layers.

In order to further investigate the composition of the TaₓTh_{y}O_{z} film, sample NC043T was prepared by depositing an ethanolic Ta(OEt)₅/Th(NO₃)₄ sol-gel onto a ZrO₂ surface formed by heat-treating a Zr disk. Using this preparation method, any Ta EDX signal would be exclusively imputable to the sol-gel derived film, and not the underlying surface. SEM imaging shows that at the boundary of the deposited area, a small amount of flaky deposits were present (See FIG. 5a, NC043T), which was due to the improper spreading of the deposited sol-gel droplet, caused by the poor wettability of the ZrO₂ surface. Most importantly, the EDX signal inside the deposition region (See FIG. 5, Area 2) showed both weak Ta and Th signals in an atomic ratio consistent with the quantities used for the preparation of the sol, as outlined in Table 2. This can be seen as further evidence that the Th atoms are homogenously mixed in the deposited film, and do not form individual particles.

TABLE 2
Atomic composition of edge region of sample NC043T (see
FIG. 5, Area 2) as detected by EDX spectroscopy.

| Element | Atom (%) | Abs. error (%) | |
|---|---|---|---|
| O | 56.72 | 3.14 | |
| Zr | 25.98 | 2.24 | |
| C | 15.63 | 1.09 | Ta/Th ratio |
| Ta | 0.77 | 0.39 | 1.09 |
| Th | 0.70 | 0.22 | |
| Hf | 0.20 | 0.24 | |

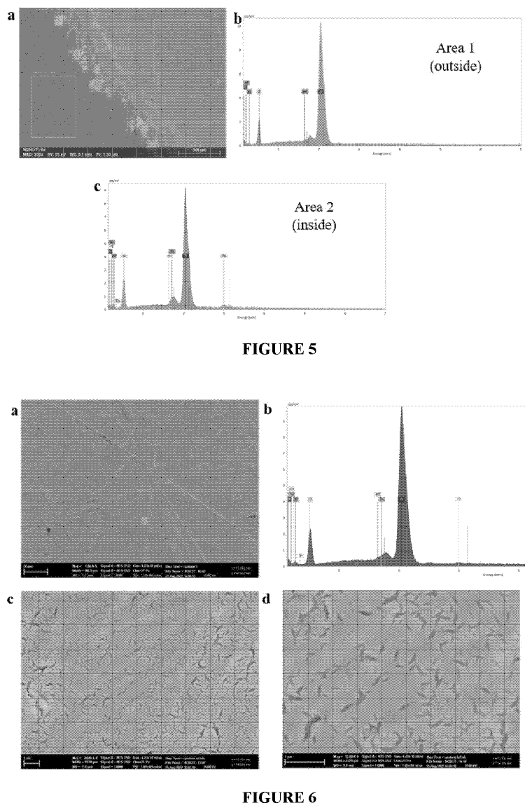

SEM images of the bulk deposition area of sample NC043T showed no discernible features at low magnification (see FIGS. 6a, 6c and 6d). The corresponding EDX spectrum shows both weak Ta and Th signals, in an atomic ratio consistent with the amounts used in the preparation of the sol-gel, as outlined in Table 3. This provides further evidence that the Th atoms are homogenously mixed in the deposited film, and that no diffusion-based concentration of the Th particles or aggregation into discrete particles is occurring.

TABLE 3
Atomic composition of bulk deposition are of sample
NC043T (See FIG. 6) as detected by EDX spectroscopy.

| Element | Atom (%) | Abs. error (%) | |
|---|---|---|---|
| O | 57.71 | 3.27 | |
| Zr | 26.70 | 2.35 | |
| C | 15.09 | 1.11 | Ta/Th ratio |
| Ta | 0.19 | 0.25 | 1.05 |
| Th | 0.18 | 0.14 | |
| Hf | 0.13 | 0.22 | |

### Example 3: Small-Batch Non-Automated Deposition Method Test (NC051T)

To better facilitate small-batch manufacture of the layered radioisotope source, application of a larger volume of the sol-gel solution is required and a small amount of aqueous nitric acid (HNO₃) is needed to efficiently extract the ²²⁸Th(NO₃)₄ from the supplied container. To assess the properties of the TaₓTh_{y}Oₓ film produced when these methodology changes were implemented, a deposition experiment using ²²⁸Th extraction using nitric acid outlined above in Example 1 was performed.

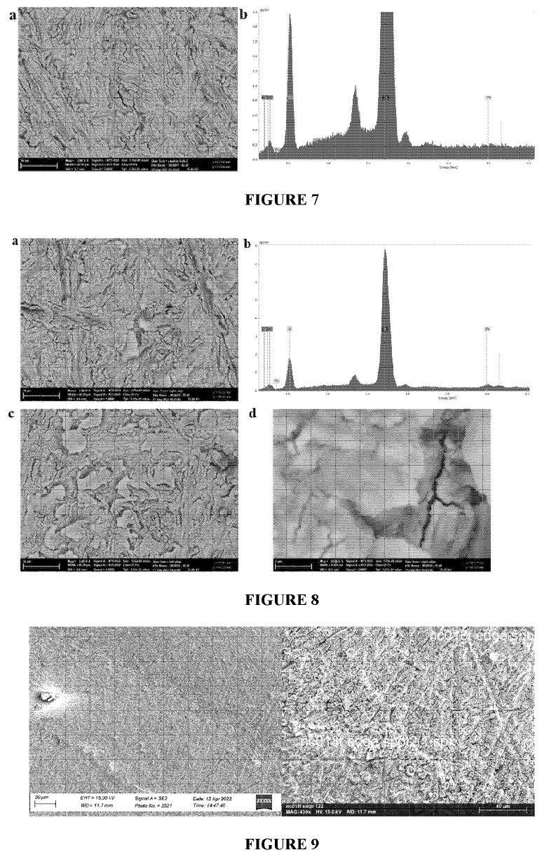

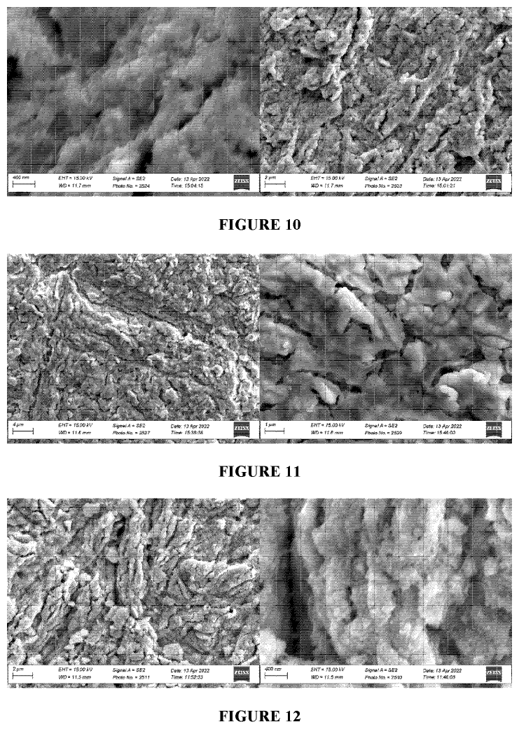

SEM images of this sample, NC051T, showed that the bulk area of the film deposited using this method presented the same relatively featureless morphology as samples NC028T and NC046T (see FIG. 7a, NC051T bulk region). A weak Th signal was detected in all bulk regions examined by SEM-EDX spectroscopy (see FIG. 7b, NC051T bulk region), with larger deviations in morphology found in deposition edge regions (see FIG. 8, NC051T edge region).

### Example 4: TaₓTh_{y}O_{z} Films Prepared Via Sol-Gel Process Comprises Tightly Bound ²²⁸Th

Layered radioisotope sources were prepared using either a tantalum disk substrate conditioned to exhibit a Ta₂O₅ surface oxide layer according to Example 1 or a planar fused quartz disk substrate pre-treated with 0.1M HNO₃.

Stock solutions of ²²⁸Th(NO₃)₄ and Ta(OEt)₅ were prepared according to Example 1. The ²²⁸Th(NO₃)₄ solution was deposited onto the surface of the substrate with or without the addition of the Ta(OEt)₅ solution followed by heat treatment at 280° C. for 2 hours. The ²²⁸Th content on each layered source was measured by alpha spectroscopy. Each source was then submerged in 0.1M HNO₃ at room temperature for 20 seconds, dried at 60° C. and the ²²⁸Th content re-measured by alpha spectroscopy. The resulting loss of ²²⁸Th from the source was calculated from the integrated value of the ²²⁸Th peak in the alpha spectrum.

| | | ²²⁸Th alpha | | |
|---|---|---|---|---|
| | | spectrum counts | | |
| | | Before | After | |
| Source | Treatment | treatment | treatment | % ²²⁸Th loss |
| Quartz source | Submersion in | 2336 | 232 | 90% |
| without tantalum | HNO3 | | | |
| alkoxide | | | | |
| Tantalum source | Submersion in | 4173 | 1048 | 75% |
| without tantalum | HNO₃ | | | |
| alkoxide | | | | |
| Quartz source | Submersion in | 3030 | 1504 | 50% |
| with tantalum | HNO3 | | | |
| alkoxide | | | | |
| Tantalum source | Submersion in | 3931 | 2765 | 30% |
| with tantalum | HNO₃ | | | |
| alkoxide | | | | |

The addition of Ta(OEt)₅ together with ²²⁸Th(NO₃)₄ resulting in the formation of a TaₓTh_{y}O_{z} surface layer on the source via the sol-gel process according to Example 1 significantly increases the retention of ²²⁸Th upon submersion in 0.1M HNO₃—which is effectively an acid etch of the surface. Even under such harsh conditions, sol-gel derived TaₓTh_{y}O_{z} surface layers comprising ²²⁸Th retained 50% or more of ²²⁸Th. This holds true whether the substrate underlying the TaₓTh_{y}O_{z} surface layer is Ta/Ta₂O₅ or quartz.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A layered radioisotope source for generating a medically useful dose of daughter radioisotope through a chain of spontaneous decay from a parent radioisotope via a gaseous intermediate radioisotope, comprising:
an inert ceramic substrate layer;
a metal oxide surface layer bound on the inert ceramic substrate layer; and
parent radioisotope immobilised on or within the metal oxide surface layer to allow for effective emanation of the gaseous intermediate radioisotope away from the layered radioisotope source.

2. The layered radioisotope source of claim 1, wherein the metal oxide surface layer is a sol-gel reaction product.

3. The layered radioisotope source of claim 1 or claim 2, wherein the metal oxide surface layer is a calcined metal oxide surface layer.

4. The layered radioisotope source of any one of claims 1 to 3, wherein the metal oxide surface layer comprises less than about 1000, 5000, 1000, 500, 100, 80, 60, 40, 20, 10, 5 or 1 discrete deposits of the radioisotope having a particle size of about 20 nm or more bound on or within the surface of the metal oxide surface layer per cm² of metal oxide surface layer.

5. The layered radioisotope source of any one of claims 1 to 3, wherein the metal oxide surface layer is substantially free of discrete deposits of the radioisotope having a particle size of about 20 nm or more bound on or within the surface of the metal oxide surface layer.

6. The layered radioisotope source of any one of claims 1 to 5, wherein the immobilised parent radioisotope are uniformly distributed within the metal oxide surface layer.

7. The layered radioisotope source of any one of claims 1 to 6, wherein the metal oxide surface layer has a thickness of between about 0.1 nm to about 1000 nm.

8. The layered radioisotope source of any one of claims 1 to 7, wherein the metal oxide surface layer has molar ratio of metal forming the oxide of the surface layer to immobilised parent radioisotope (M: R) of between greater than 1 to about 10.

9. The layered radioisotope source of any one of claims 1 to 8, wherein the metal oxide surface layer is an oxide of a valve metal, a refractory metal or another transition or main-block metal.

10. The layered radioisotope source of any one of claims 1 to 9, wherein the metal oxide surface layer is an oxide of tantalum, niobium, tungsten, molybdenum, vanadium, zirconium, or titanium, or a mixed oxide thereof.

11. The layered radioisotope source of any one of claims 1 to 10, wherein the metal oxide surface layer is tantalum pentoxide (Ta₂O₅)

12. The layered radioisotope source of any one of claims 1 to 11, wherein the metal oxide surface layer and at least some of the immobilised parent radioisotope together form one or more mixed oxide phases within the metal oxide surface layer.

13. The layered radioisotope source of any one of claims 1 to 12, wherein the metal oxide surface layer and immobilised parent radioisotope form a mixed oxide surface layer on the inert ceramic substrate.

14. The layered radioisotope source of claim 12 or claim 13, wherein the mixed oxide phase or mixed oxide surface layer has the formula RₓM_{y}O_{z}, wherein R is a parent radioisotope in cationic form, M is one or more metals in cationic form, 0.1≤x≤5, 1.0≤y≤20, 1.0≤z≤50, preferably wherein x<y.

15. The layered radioisotope source of any one of claims 1 to 14, wherein the inert ceramic substrate layer has a thickness (in μm) of between about 1 to about 1000.

16. The layered radioisotope source of any one of claims 1 to 15, wherein the inert ceramic substrate layer is selected from an inert oxide, an inert nitride, an inert carbide, an inert sulfide, an inert phosphate or combination thereof.

17. The layered radioisotope source of any one of claims 1 to 16, wherein the inert ceramic substrate layer is selected from a metal oxide, metal nitride, metal carbide, metal sulfide, metal phosphate or combination thereof.

18. The layered radioisotope source of any one of claims 1 to 17, wherein the inert ceramic substrate layer is an oxide of silicon, tantalum, niobium, tungsten, molybdenum, vanadium, zirconium, titanium or aluminium, or mixed oxides thereof.

19. The layered radioisotope source of any one of claims 1 to 18, wherein the inert ceramic substrate layer is quartz, tantalum pentoxide (Ta₂O₅) or zirconium dioxide (ZrO₂).

20. The layered radioisotope source of any one of claims 1 to 19, wherein the inert ceramic substrate layer is provided on a metal substrate.

21. The layered radioisotope source of claim 20, wherein the metal substrate is selected from the group consisting of tantalum, niobium, tungsten, hafnium, molybdenum, vanadium, zirconium, titanium or aluminium, or alloys thereof.

22. The layered radioisotope source of claim 20 or claim 21, wherein the inert ceramic substrate layer is produced by oxidatively pre-treating the surface of the metal substrate.

23. The layered radioisotope source of any one of claims 20 to 22, wherein the metal substrate has a density (in g/cm³) of between about 2.0 to about 20.

24. The layered radioisotope source of any one of claims 20 to 23, wherein the metal substrate has a thickness (in mm) of between about 1 to 100.

25. The layered radioisotope source of any one of claims 1 to 24, wherein the immobilised parent radioisotope within the metal oxide surface layer is such that, in use, it enables the capture of a population of daughter radioisotope having a contamination level of parent radioisotope of less than about 5% expressed in activity terms relative to the activity of the daughter radioisotope.

26. The layered radioisotope source of any one of claims 1 to 25, wherein the layered radioisotope source is provided as a disk or slab.

27. The layered radioisotope source of any one of claims 1 to 26, wherein the layered radioisotope source has a thickness (in mm) of between about 1 to 100.

28. The layered radioisotope source of any one of claims 1 to 28, wherein the parent radioisotope is an alpha-emitting radioisotope.

29. The layered radioisotope source of any one of claims 1 to 28, wherein the parent radioisotope is a thorium radioisotope selected from thorium-227 (²²⁷Th) or thorium-228 (²²⁸Th).

30. The layered radioisotope source of any one of claims 1 to 29, wherein the daughter radioisotope is a lead radioisotope selected from at least one of lead-211 (²¹¹Pb) or lead-212 (²¹²Pb).

31. The layered radioisotope source of any one of claims 1 to 30, wherein the immobilised parent radioisotope is present in an amount effective to provide an activity (in MBq per cm² of inert ceramic substrate surface) of between about 1 to about 1500.

32. A sol-gel process for preparing a layered radioisotope source for generating a medically useful dose of daughter radioisotope through a chain of spontaneous decay from a parent radioisotope via a gaseous intermediate radioisotope, comprising:
a) providing on the surface of an inert ceramic substrate layer a gel formed from a solution (e.g. sol) comprising a metal alkoxide and a parent radioisotope:
b) heating the gel under conditions effective to form a metal oxide surface layer bound on the inert ceramic substrate layer,
wherein the parent radioisotope is immobilised on or within the metal oxide surface layer to allow for effective emanation of the gaseous intermediate radioisotope away from the layered radioisotope source.

33. The sol-gel process of claim 32, wherein the process is for preparing a layered radioisotope source of any one of claims 1 to 31

34. The sol-gel process of claim 32 or claim 33, wherein after step a) but prior to step b), the gel is dried.

35. The sol-gel process of claim 34, wherein the gel is dried at a temperature (in ° C.) of between about 10 to about 180, and preferably for a period of time (in hours) of between about 0.1 to about 100.

36. The sol-gel process of any one of claims 32 to 35, wherein the solution at step a) comprises an alcoholic solution of the metal alkoxide and parent radioisotope species.

37. The sol-gel process of claim 36, wherein the alcoholic solution of metal alkoxide and parent radioisotope species comprises an alcohol solvent in amount (in % v/v based on the total volume of the solution) of between about 50 to 99.

38. The sol-gel process of any one of claims 32 to 37, wherein the metal alkoxide is in stoichiometric excess relative to the parent radioisotope species.

39. The sol-gel process of any one of claims 32 to 38, wherein molar ratio of metal alkoxide to parent radioisotope species is between greater than 1 to about 10.

40. The sol-gel process of any one of claims 32 to 39, wherein the parent radioisotope species is provided in the solution at a concentration of between about 0.000001 M to about 0.01 M.

41. The sol-gel process of any one of claims 32 to 40, wherein the metal alkoxide is provided in the solution at a concentration of between about 0.000050 M to about 0.01 M.

42. The sol-gel process of any one of claims 32 to 41, wherein the solution further comprises water and/or an acid.

43. The sol-gel process of claim 42, wherein the acid is provided in the solution at a concentration of between about 0.1 M to about 1 M

44. The sol-gel process of any one of claims 32 to 43, wherein the gel is heated at step b) at a temperature (in ° C.) of between about 200 to about 500, and preferably for a period of time (in minutes) of between about 30 to about 360.

45. The sol-gel process of any one of claims 32 to 44, wherein the metal alkoxide is an alkoxide of a valve metal, a refractory metal or another transition or main-block metal.

46. The sol-gel process of any one of claims 32 to 45, wherein the metal alkoxide is an alkoxide of tantalum, niobium, tungsten, molybdenum, vanadium, zirconium, or titanium, or a mixed oxide thereof.

47. The sol-gel process of any one of claims 32 to 46, wherein the metal alkoxide is tantalum alkoxide, preferably tantalum ethoxide.

48. The sol-gel process of any one of claims 32 to 47, wherein the parent radioisotope species is provided as a salt or hydrate thereof selected from one or more of hydroxides, halides, phosphates, nitrates, acetates, sulfates, perchlorates, ammonium compounds and anionic oxo-metallate compounds.

49. The sol-gel process of any one of claims 32 to 48, wherein the parent radioisotope species is a thorium species.

50. The sol-gel process of claim 49, wherein the thorium species is a nitrate salt or hydrate thereof.

51. A radioisotope generator defining a chamber for producing and capturing a population of daughter radioisotope, wherein the chamber is configured to house a layered radioisotope source of any one of claims 1 to 31 in the chamber.

52. The radioisotope generator of claim 51, wherein the chamber comprises a collection surface and is configured to house the layered radioisotope source in the chamber with the metal oxide surface layer facing the collection surface for collecting at least some of the emanated gaseous intermediate radioisotope for a period of time effective for it to decay into daughter radioisotope.

53. The radioisotope generator of claim 52, wherein the metal oxide surface layer is in line-of-sight configuration with the collection surface.

54. The radioisotope generator of claim 52 or claim 53, wherein the metal oxide surface layer substantially faces downwards to enable gravity assisted collection of at least some of the emanated gaseous intermediate radioisotope on the collection surface.

55. The radioisotope generator of any one of claims 52 to 54, further comprising a carrier gas inlet port configured to introduce a carrier gas into the chamber to facilitate transfer of emanated gaseous intermediate radioisotope away from the layered radioisotope source onto the collection surface.

56. The radioisotope generator of any one of claims 52 to 55, further comprising a vacuum pump configured to apply a vacuum and evacuate the chamber to facilitate transfer of emanated gaseous intermediate radioisotope away from the layered radioisotope source onto the collection surface.

57. The radioisotope generator of any one of claims 52 to 56, further comprising a fluid delivery system configured to introduce a collection fluid into the chamber to collect daughter radioisotope from the collection surface.

58. The radioisotope generator of claim 57, further comprising a collection fluid outlet port configured to transfer the collection fluid comprising daughter radioisotope from the chamber.

59. A system for producing and capturing a population of daughter radioisotope, comprising:
a) a radioisotope generator defining a chamber for producing and capturing a population of daughter radioisotope; and
b) a layered radioisotope source of any one of claims 1 to 31 housed in the chamber.

60. The system of claim 59, comprising a radioisotope generator of any one of 51 to 57.

61. A process for producing and capturing a population of daughter radioisotope comprising:
a) allowing for the emanation of a gaseous intermediate radioisotope generated through a chain of spontaneous decay from a parent radioisotope immobilised on or within a layered radioisotope source of any one of claims 1 to 31, and
b) collecting at least some of the gaseous intermediate radioisotope for a period of time effective for it to decay into a daughter radioisotope.

62. The process of claim 61, comprising a radioisotope generator of any one of claims 51 to 57, or a system of claim 59 or claim 60.

63. The process of claim 61 or claim 62, wherein the process further comprises a step of recovering at least some of the daughter radioisotope.

64. The process of claim 63, wherein the recovered daughter radioisotope is conjugated to a targeting molecule for use in radioligand therapy.

65. The process of claim 64, wherein the targeting molecule is a cancer cell targeting molecule.

66. The process of any one of claims 61 to 65, wherein the parent radioisotope is an alpha-emitting radioisotope.

67. The process of any one of claims 61 to 66, wherein the parent radioisotope is a thorium radioisotope selected from at least one of thorium-227 (²²⁷Th) and thorium-228 (²²⁸Th).

68. The process of any one of claims 61 to 67, wherein the gaseous intermediate radioisotope is a radon radioisotope selected from at least one of radon-219 (²¹⁹Rn) and radon-220 (²²⁰Rn).

69. The process of any one of claims 61 to 68, wherein the daughter radioisotope is a lead radioisotope selected from at least one of lead-211 (²¹¹Pb) or lead-212 (²¹²Pb).
